SEQUENCE LISTING (2 sequences)

SEQ ID NO: 1 - DNA, 20 bp, synthetic construct
  source 1..20: mol_type = other DNA
tatgaccgagcacttcccct

SEQ ID NO: 2 - DNA, 20 bp, synthetic construct
  source 1..20: mol_type = other DNA
tcagagacaggttatgccgc

(19) United States
(12) Patent Application Publication
(10) Pub. No.: US 2026/0275290 A1
(43) Pub. Date: Sep. 17, 2026

(54) METHODS FOR GENERATING ARTIFICIAL THYMUS, PRODUCTS AND USES THEREOF

(71) Applicants: GENEWITY B.V., Oegstgeest (NL); LEIDS UNIVERSITAIR MEDISCH CENTRUM (LUMC), Leiden (NL)

(72) Inventors: Frank Jakob Theodor STAAL, The Hague (NL); Aline ZBINDEN, Leiden (NL); Henricus Johannes Hermanus Maria CLASSEN, Lelystad (NL); Arno BISSCHOP, Amsterdam (NL); Max Jurie RENES, Utrecht (NL)

(21) Appl. No.: 19/167,248

(22) PCT Filed: Mar. 20, 2024
(86) PCT No.: PCT/EP2024/057477

§ 371 Date: Sep. 19, 2025

(30) Foreign Application Priority Data
Mar. 21, 2023 (EP) 23163148.2

(51) Int. Cl. C12N 5/078; A61K 35/26; C07K 14/00; C12N 5/00; C12N 5/071; C12N 5/077; C12N 5/0783; C12N 15/867

(52) U.S. Cl. CPC C12N 5/065; A61K 35/26; C07K 14/00; C12N 5/0062; C12N 5/0636; C12N 5/0656; C12N 5/0697; C12N 15/867; C12N 2501/15; C12N 2501/155; C12N 2506/45; C12N 2510/00; C12N 2513/00; C12N 2740/15043; C12N 2830/003; C12N 2830/34; C12N 2840/44

(57) ABSTRACT

The invention provides a method for producing a three dimensional thymic like structure, the method comprising: a) providing pluripotent cells from a recipient with a nucleic acid molecule comprising a FOXN1 sequence, and b) inducing said pluripotent cell to differentiate into thymic epithelium in a scaffold suitable for transplantation, as well as related methods, compositions or uses.

### BACKGROUND

The invention relates to the field of genetic diseases. In particular, the invention relates to genetic defects resulting in immune deficiencies. More in particular, the invention relates to immune deficiencies as a result of a (fully) dysfunctional thymus. The invention provides means and methods for providing a thymus-like environment, where immune cells can develop in situ.

Good examples of the field of the invention are inborn deficiencies in thymus development. Inborn errors of thymic stromal cell development and function lead to impaired T-cell development resulting in a susceptibility to opportunistic infections and autoimmunity. In their most severe form, congenital athymia, these disorders are life-threatening when left untreated. Athymia is rare and is typically associated with complete DiGeorge syndrome, which has multiple genetic and environmental etiologies. It is also found in rare cases of T-cell lymphopenia due to Nude SCID and Otofaciocervical Syndrome type 2, or in the context of genetically undefined defects. This group of disorders cannot be corrected by hematopoietic stem cell transplantation, but upon timely recognition as thymic defects, can be treated by thymus transplantation using cultured postnatal thymic tissue with the generation of naïve T-cells that hold a diverse repertoire. The mortality post-treatment relates usually to adverse events that occurred before reaching immune reconstitution and is mainly associated with infections that are most often acquired pre-transplantation. Notwithstanding, transplantation comes with all its known drawbacks of having to at least partially match donor and recipient, the general lack of available donor material, in particular at the time required. The present invention is directed at solving these problems (at least partially) by providing thymus-like structures that can be transplanted and are autologous to the recipient of said structures.

### SUMMARY OF THE INVENTION

The invention thus provides means and methods for producing a three dimensional thymic-like structure, comprising differentiated pluripotent cells that contain a nucleic acid molecule comprising a FOXN1 (Forkhead box protein N1) sequence and inducing said pluripotent cell to differentiate into thymic epithelium and/or into a thymus-like structure. Said thymus material can be transplanted in a recipient, upon which immune cells of the recipient can develop and/or mature in the context of said thymus-like material.

In one aspect, the invention provides a method for producing a three dimensional thymic like structure, the method comprising: a) providing pluripotent cell(s) from a recipient with a nucleic acid molecule comprising a FOXN1 sequence, and b) inducing said pluripotent cell(s) to differentiate into thymic epithelium in a scaffold suitable for transplantation.

In one aspect, the invention provides a method for producing a three dimensional thymic like structure, the method comprising: a) providing pluripotent cell(s) from a recipient, and b) inducing said pluripotent cell(s) to differentiate into thymic epithelium in a scaffold suitable for transplantation,
wherein a nucleic acid molecule comprising a FOXN1 sequence is provided to the pluripotent cell(s) from a recipient of a) or to cells of a differentiation stage in b) selected from definitive endoderm cells, anterior foregut endoderm cells, and pharyngeal pouch endoderm cells.

In embodiments, said scaffold comprises cells derived from the recipient and/or HLA-matched with such recipient.

In one aspect, a nucleic acid molecule comprising a FOXN1 sequence is provided, wherein the nucleic acid further comprises nucleic acid sequences for introducing said FOXN1 sequence in a pluripotent cell.

In embodiments, the nucleic acid further comprises nucleic acid sequences for introducing said FOXN1 sequence in a safe harbour locus of a pluripotent cell.

In one aspect, a nucleic acid molecule comprising a FOXN1 sequence is provided, wherein the nucleic acid further comprises nucleic acid sequences for introducing said FOXN1 sequence in a safe harbour locus of a pluripotent cell.

In one aspect, a vector is provided comprising a nucleic acid molecule provided herein.

In embodiments, the nucleic acid sequences for introducing a FOXN1 sequence in a safe harbour locus of a pluripotent cell comprise homologous sequences to a safe harbour locus.

In embodiments, the safe harbour locus is Adeno-associated virus integration site 1 (AAVS1), the chemokine (C-C motif) receptor 5 (CCR5), or human Rosa26.

In embodiments, the vector remains episomal upon introduction to a pluripotent cell.

In aspects, a viral vector comprising a vector provided herein and a viral capsid is provided.

In embodiments, the viral vector is of lentiviral origin.

In aspects, a method for producing a thymic epithelial progenitor cell (TEPC) from a pluripotent cell is provided comprising the steps: providing said pluripotent cell with a nucleotide vector provided herein, thereby obtaining a transduced cell, and subjecting said transduced cell to a differentiation protocol, optionally wherein the differentiation protocol comprises stepwise and timed modulation of key developmental pathways including the Wnt, SHH (Sonic hedgehog), TGFB (transforming growth factor beta), BMP (Bone morphogenetic protein) and/or retinoic acid signaling pathways.

In aspects, a method for producing a TEPC from a pluripotent cell is provided comprising the steps: infecting a pluripotent cell with a viral vector provided herein, thereby obtaining a transduced cell, and subjecting said transduced cell to a differentiation protocol, optionally wherein the differentiation protocol comprises stepwise and timed modulation of key developmental pathways including the Wnt, SHH, TGFB, BMP and/or retinoic acid signaling pathways.

In embodiments of methods herein, the pluripotent cell differentiates into TEPC via the intermediate stages of definitive endoderm cells, anterior foregut endoderm cells, and pharyngeal pouch endoderm cells and/or wherein the pluripotent cell differentiates into definitive endoderm cells and the differentiation of definitive endoderm to anterior foregut endoderm is induced when the percentage of SP (single positive) CD117+ is at least 30% and the percentage of DP (double positive) CD117+CD184+ cells is at most 70%.

In embodiments, the differentiation of definitive endoderm to anterior foregut endoderm is induced by exposing the cells to dual TGFβ/BMP inhibition, in particular dual TGFβ/BMP4 inhibition.

In embodiments, FOXN1 expression is induced at the pharyngeal pouch endoderm stage.

In aspects, a TEPC comprising a FOXN1 encoding sequence integrated in its DNA is provided.

In embodiments, said TEPC comprising a FOXN1 encoding sequence comprises said FOXN1 encoding sequence integrated in its DNA in a safe harbour locus.

In embodiments, said TEPC comprising a FOXN1 encoding sequence comprises said FOXN1 encoding sequence randomly integrated in its DNA.

In embodiments, the thymus organoid is of human origin.

In embodiments, the cells of the thymus organoid are of human origin.

In embodiments herein, the safe harbour locus is AAVS1, CCR5, or humanRosa26.

In embodiments, the FOXN1 encoding sequence is under the control of an inducible promoter.

In embodiments, the inducible promoter is a tetracycline-inducible promoter (TET on or TET off promoter), an estrogen inducible promoter (e.g. a promoter inducible by estradiol or analogues thereof or by tamoxifen, a cumate-controlled promotor (e.g. VP16), a FKBP12 and/or cyclophilin inducible promoter, a FRAP (protein-protein interaction induced by small molecule) promoter, or a metallothionein promoter.

In a further aspect, a method for inducing a thymic epithelial cell (TEC) from a TEPC as defined herein is provided, the method comprising contacting said TEPC with an autologous hematopoietic stem cell or autologous hematopoietic progenitor cell or an autologous common lymphoid progenitor or an autologous T-cell progenitor under conditions allowing for said induction, optionally wherein said contacting comprises co-culturing said TEPC with an autologous hematopoietic stem cell or an autologous common lymphoid progenitor or an autologous T-cell progenitor.

In a further aspect, a thymus epithelial cell (TEC) obtainable by a method for inducing a TEC from a thymic epithelial progenitor cell (TEPC) is provided.

In a further aspect, a thymus spheroid or organoid comprising at least one TEPC provided herein and/or at least one TEC provided herein, is provided.

In a further aspect, a spheroid or organoid herein is provided wherein the spheroid or organoid further comprising supporting cells.

In embodiments, the supporting cells comprise mesenchymal stromal cells (MSCs).

In embodiments, the supporting cells comprise fibroblasts.

In embodiments, the supporting cells share their origin with the TEPCs and/or TECs.

In embodiments, the spheroid or organoid is further comprising a supporting matrix that comprises Collagen type 1, hydrogels based on fibronectin, laminins, and other proteoglycans, alginate, hyaluronic acid-based gels, or Hystem, or a combination of at least any two thereof.

In embodiments, the supporting matrix comprises 3D printed materials.

In embodiments, the thymus spheroid or organoid has a medulla-like and/or cortex-like three-dimensional structure.

In aspects, provided herein is a spheroid or organoid provided herein for use in cell therapy.

In aspects, provided herein is a spheroid or organoid provided herein for use in the treatment of congenital and/or acquired diseases.

In aspects, provided herein is a spheroid or organoid provided herein for use in the treatment of an immune deficiency.

In aspects, provided herein is a spheroid or organoid provided herein for use in the treatment of a lack of a functional thymus.

In aspects, provided herein is a spheroid or organoid provided herein for use in the treatment of DiGeorge syndrome.

In aspects, provided herein is a spheroid or organoid provided herein for use in the treatment of DiGeorge syndrome, Otofaciocervical Syndrome, or CHARGE syndrome.

In aspects, provided herein is a spheroid or organoid provided herein for use in the treatment of FOXN1 deficiency.

In aspects, provided herein is a spheroid or organoid provided herein for use in the treatment of a lack of a fully functional immune repertoire as a result of chemotherapy.

In aspects, provided herein is a spheroid or organoid provided herein for use in the treatment of a lack of a fully functional immune repertoire as a result of hematopoietic stem cell transplantation.

In aspects, provided herein is a spheroid or organoid provided herein for use in the treatment of a lack of a functional thymus as a result of thymectomy.

In aspects, provided herein is a spheroid or organoid provided herein for use in the treatment of a lack of a functional thymus as a result of thymus involution.

In embodiments herein, the spheroid or organoid is implanted in skeletal muscle.

In embodiments herein, the skeletal muscle is a quadriceps muscle.

In embodiments herein, said skeletal muscle shares its origin with said spheroid or organoid.

In embodiments of various aspects herein, the FOXN1 sequence is codon optimized for human codon usage, and wherein said codon optimization comprises removal of at least one CpG island and/or at least one cryptic splice site.

In an aspect, a method of producing at least one T cell or T lineage cell is provided, wherein the method comprises the steps of: a) providing a spheroid or organoid as specified herein comprising at least one thymus epithelial cell (TEC) and/or thymus epithelial progenitor cell (TEPC), b) contacting the spheroid or organoid of a) with at least one hematopoietic progenitor cell or T lineage cell, thereby producing at least one T cell or T lineage cell.

### FIGURE LEGEND

FIG. 1. Characterization of an induced pluripotent stem cells (iPSC) line containing a Doxycycline (DOX)-inducible construct for coFOXN1. A. Schematic of the construct: generation of the 3G protein from its encoding sequence (depicted as “Tet-On 3G”) is under the control of an MND promoter, as exemplary constitutive promoter. This provides an exemplary construct using a 3^{rd} generation tetracycline-responsive Tet-On system as inducible promoter system. Under the same promoter is also a puromycine resistance gene for selection purpose. It is understood that also other suitable resistance markers can be used. The addition of the tetracycline doxycycline allows the 3G protein, which is a transactivator protein, to bind to its promoter (TRE3G) in the presence of doxycycline, leading to the transcription of human codon-optimized FOXN1 gene (coFOXN1; depicted as “co-Hu-FOXN1” in scheme A. Under the same DOX-responsive promoter, the reporter gene mScarlet is also transcribed. The role of the T2A-P2A sequence is to induce a “jump” during the translation by ribosomes in order to express the FOXN1 protein and the reporter (here: mScarlet) as distinct, separate proteins. B. Analysis of pluripotency markers Oct3/4 and SOX2 in two coFOXN1 clones (coFOXN1mSc-2 and coFOXN1mSc-4) and an iPSC control (LUMC0044iCtrl44). The expression of pluripotency markers Oct3/4 and SOX2 was found to be similar to the expression in the iPSC control, indicating that the introduction of the construct in the AAVS1 locus has not altered the pluripotency of the iPSC line. C. Gene expression analysis by qPCR showing the presence of coFOXN1 at the mRNA level when coFOXN1 iPSCs are treated with doxycycline. D. Brightfield and fluorescent images of a coFOXN1mSc iPSC colony with doxycycline (+Dox). E. Expression of mScarlet reporter protein in two coFOXN1mSc iPSC clones (coFOXN1mSc-2 and coFOXN1mSc-4) with doxycycline (+Dox) and without doxycycline (−DOX), as measured by spectral flow cytometry (FC). The intensity is also compared to the unmodified iPSC line (control). A clear reporter signal was observed with doxycycline. Close to 100% of the coFOXN1mSc cells are mScarlet+, when exposed to DOX. F. coFOXN1mSc cells upregulate DLL4 (Delta-like 4) mRNA when exposed to DOX at the iPSC stage (left graph) and during differentiation of the cells (right graph).

FIG. 2: Further characterization of the DOX-inducible line: proper integration within the safe harbor locus. A. Schematic of the construct with the genomic Polymerase Chain Reaction (PCR) strategy illustrated B. Genomic PCR showing the proper integration of the construct into the safe harbour locus (here: AAVS1) of an iPSC line. In this example, iPSC clones a to f are shown. The products of lanes PCR 1 show that the correct sequence (coFOXN1) is present; lanes PCR 2 indicate that the construct has been integrated into the safe harbour locus AAVS1.

FIG. 3. Comparison of two different anterior foregut endoderm (AFE) induction protocols. A. Protocol A included stimulation of Bone morphogenetic protein 4 (BMP), whereas protocol B included inhibition of BMP signaling. B. LUMC0044-iCtrl44, coFOXN1mSc-2 and coFOXN1mSc-4 have comparable levels of CD117+CD184+ double positive (DP) cells at day 5 (definitive endoderm (DE) efficiency). C. Protocol B induces higher percentages of CD56+CD271+ DP cells at day 8 for all three cell lines, which indicates improved AFE induction.

FIG. 4: Further characterization of protocol A and B: CD56+CD271+ marks SOX2+ cells. A. Schematic illustration of the two protocols A and B for the AFE stage. B. FC plot showing that CD56+CD271+ DP cells are SOX2+ at the AFE stage. SOX2 is widely used to describe AFE populations arising from definitive endoderm. C. Both FOXA2 and SOX2 expression was assessed using qPCR for the protocol A (dotted line) and protocol B (solid line). Only using protocol B, re-upregulation of SOX2 was achieved, strengthening the data that suggest the superiority of protocol B over A to generate AFE cells.

FIG. 5. Timing of definitive endoderm (DE) and anterior foregut endoderm (AFE) induction. A. Different induction timepoints for DE and AFE were tested. B. iPSCs differentiating to DE first become CD117+ single positive (SP) and then CD117+CD184+ double positive (DP) (representative graphs at day 3 and 4 of differentiation) and C. quantification. D. Representative graph showing CD56+ and CD271+ population at the AFE stage E. (left graph) The percentage of CD117+CD184+ DP cells (DE stage) and CD56+CD271+ DP cells (AFE), and (right graph) CD117+ SP cells at DE correlates positively with the percentage of CD56+CD271+ DP cells at AFE. F. Experiments with a high percentage of AFE DP cells cluster at higher expression of the AFE TFs TBX1 and PAX9.

FIG. 6. AFE induction efficiencies over multiple experiments. DE efficiencies (showed by CD117+CD184+ DP) and AFE efficiencies (CD56+ CD271+ DP) showed over 3 independent experiments.

FIG. 7: Replating DE cells at lower density to overcome low AFE efficiencies. A. Replating cells one day before DE stage doesn't affect the efficiencies of the DE stage. Representative FC plot shows >80% of CD117+ CD184+ replated cells, where DP cells for CD117+ and CD184+ are also OCT4 negative. B. Positive effect of replating cell on the SOX2 re-upregulation with DE cells of high percentage (more than 80%). C-D TBX1 and FOXA2 are stable in replated cells.

FIG. 8: AFE protocol B reached higher percentage of AFE cells with SB431542 (SB) and Noggin. A. FC plot showing CD117+ CD184+ DP cells close to 90% at DE stage treated with B. SB and Noggin which are alternative reagents to LY364947 (LY) and Dorsomorphin blocking the TGFB receptor type I and BMP pathway respectively. More than 90% of FOXA2+ SOX2+ cells were detected at the AFE stage. FOXA2+SOX2+ cells are commonly used to defined AFE stage within the scientific community. Together, these data show an optimized differentiation protocol through high percentage DE and AFE cells.

FIG. 9. Strategy and characterization of the generated induced thymic epithelial cell progenitor cells (iTEPCs). A. Schematic illustrating the different steps for the differentiation of iPSCs to iTEPCs. DE: definitive endoderm stage; AFE: anterior foregut endoderm; PPE: pharyngeal pouch endoderm. B. Protein and gene expression analysis of key marker genes: TBX1 (required for pouch formation), PAX9 (AFE), PAX1 (AFE), EYA1 (TEPC), HOXA3 (3rd pouch formation), FOXN1 (TEPC) and DLL4 (TEPC, required to drive T-cell differentiation). (C; left panel) EpCAM (Epithelial cell adhesion molecule) and PDPN (podoplanin) are known to be expressed on postnatal TECs in the human thymus. (C; three most right panels) During differentiation from iPSC to iTEPC, two different populations emerged based on the expression of surface markers EpCAM and PDPN.

FIG. 10: High reproducibility of the iTEPC protocol: combination of more than 10 independent differentiations. A. Schematic illustrating the different steps for the differentiation of iPSCs to iTEPCs. DE: definitive endoderm stage; AFE: anterior foregut endoderm; PPE: pharyngeal pouch endoderm. B. Protein and gene expression analysis of key marker genes: OCT4 is a marker pluripotency (iPSC stage), SOX17 together with CD117 and CD184 markers for definitive endoderm stage (DE). FOXA2 is a marker for DE and anterior foregut endoderm (AFE) stage. HOXA3 is a marker for PPE stage. FOXN1 is a specific marker for the thymic progenitor cells. In addition, DLL4 expression increased during differentiation, which is a target of FOXN1 and a determinant to support T-cell development. C. Comparison of podoplanin and EpCAM expression in the human neonatal thymus and in the iTEPCs. iTEPCs also express CKT8 (cytokeratin-8) which is a key TEPC marker.

FIG. 11. Comparison of different PPE induction media. A. Schematic representation of the different 2-day and 4-day media tested. B. Medium D/H (TEC induction medium after AFE) caused absence of HOXA3, accompanied by lower levels of FOXN1 and DLL4 for both the 2-day and 4-day medium. C. Medium D/H also resulted in lower percentages of EpCAM^{int} and EpCAM^{hi} populations. This data suggests that media A, B, C and E, F, G are preferred over D and H.

FIG. 12. Effects of coFOXN1 induction during differentiation of iPSCs to iTEPCs. A. Cells were exposed to DOX during the last stage of differentiation. B. Exposure to DOX during the last stage of differentiation induced expression of mScarlet and C. coFOXN1 mRNA. D. From the first two experiments, no major changes were observed in endogenous FOXN1 and HOXA3 expression. E. An increase in DLL4 expression have resulted from exposure to DOX. F. No major changes in CD205 expression were observed by flow cytometry, while a minor increase in DLL4 expression upon exposure to DOX was detected. DOX exposure positively impacts DLL4 expression, without negatively impacting other TEC markers.

FIG. 13: Expression of PDPN and EPCAM at the iTEPC stage, with and without DOX. Exposure to DOX during the last stage of differentiation from PPE to iTEPCs does not have negative effects on the expression of PDPN and EpCAM. Shown are the graphs from two independent experiments (A and B).

FIG. 14: Technical replicates show DOX-induction consistently increased DLL4 and coFOXN1 expression. Induction of coFOXN1 (reported by mScarlet) during differentiation is beneficial, as the induced transcription and expression of coFOXN1mSc at the PPE stage leads to DLL4 expression. These data indicate that coFOXN1 can activate its target genes during the PPE to TEPC stage. Student t-test p-value <0.05.

FIG. 15: 3D-culture of iTEPCs improves DLL4 expression. A. Schematic of the 3D culture set-up. Briefly, spheroids are formed during differentiation from AFE to PPE and are then transferred on an air-liquid interface (AL) into Geltrex™ for the last differentiation stage to induce differentiation to TEPC. B. coFOXN1 expression is induced with DOX in 3D culture, as indicated by mScarlet, and does not affect viability of the TEPCs. C. 3D culture leads to higher level of DLL4 expression in the iTEPCs as compared to 2D culture (TEC #22, TEC #27DE3, TEC #27DE5 and TEC #35 are four independent experiments). DLL4 is essential for T-cell development. In the right graph, the respective left column shows the results with 2D culture, and the respective right columns show the results with 3D culture.

FIG. 16. Optimization of the Artificial Thymic Organoid (ATO) system to test the functionality of generated iTEPCs. A. Schematic of the process of forming ATO. B. Classical MS5-hDLL1 (MS5—human Delta Like Canonical Notch Ligand 1) ATOs support survival of HPCs (by expression of CD45) and differentiation to the T-cell lineage (by expression of CD7 and CD5), also when made in Geltrex™ basement matrix components. C. iTEPC-ATOs with Geltrex™ has a higher viability of HPCs (hematopoietic progenitor cells).

FIG. 17: Robust HPC survival and emergence of CD5+CD7+ T-Cells is achieved using SCF in the ATO-like system. CD5 and CD7 are early T cell development markers. A. Schematic of iATO formation (ATO using iTEPCs). Human CD34+ cells and iTEPCs on day 14 or day 16 of differentiation from iPSCs are mixed at a ratio of 1:3 and placed onto an air-liquid interface. B. An iATO proliferates and forms defined 3D structures. C. Pooled data set and quantification showing high viability, mScarlet+ and CD45+ cells up to 4 weeks after iATO formation. After 4 weeks (W4), we observed CD33+ (myeloid population). Gating on CD33−, a T-cell progenitors CD7+CD5+ population under +DOX conditions is observed, demonstrating functionality of iTPECs.

FIG. 18: In vivo proof of concept using Balb/C nude mice. (A) Schematic overview of the pre-clinical study. Balb/C nude mice (foxn1 double knockout; “nude (foxn1−/−)”) received either sham treatment or subcapsular kidney transplantations with either organoids consisting of only MSCs (human mesenchymal stromal cells from bone marrow aspiration) (n=6 per kidney) or with iTEPC organoids expressing coFOXN1 (n=6 per kidney). (B) Visualization of the iTEPC organoid without (left) or with (right) MSCs included. (C) Frequency of the total CD3+ TCRβ+ cells (left), CD3+ TCRβ +CD4+ cells (middle) and CD3+ TCRβ +CD8+ cells (right) among the total population of immune cells, found in blood over time. (D) Representative flow cytometry plots depicting the population of CD3+ TCRβ +CD4+ cells and CD3+ TCRβ +CD8+ cells for mice transplanted with only MSC-containing organoids (upper row) and iTEPCs (lower row). Error bars indicate +1SD, N=2.

FIG. 19: In vivo proof of concept using humanized NSG nude (FOXN1−/−) mouse model. A. Schematic of the pre-clinical study. Following conditioning with Busulfan, mice (NSG-nude (foxn1−/−) were humanized with CD34+ hematopoietic stem cells (“CD34+”) two weeks (“W-2”) prior to subcapsular renal transplantation of the right kidney, using n=9 thymus organoids/per kidney comprising MSCs, iTEPCs and primed CD34+ (see methods). Priming of CD34+ was performed for 3 days according to protocol described by Montel-Hagen, Amélie, et al. (Cell reports 33.4 (2020)). At week 7 post kidney transplant, the kidney was harvested and stained with H&E (hematoxylin & eosin) to localize the organoid (O) from the kidney (K) and its capsule (C), as well as CD3 as marker for T-cell development. Clear CD3 staining was observed within the organoid. Scale bar 100 μm.

### DETAILED DESCRIPTION

Uniquely among hematopoietic stem cell derived lineages, T-cells require a second site for their development, namely the thymus. Thus, primary immunodeficiency disorders (PIDs) leading to T-cell deficiency may be a result of either hematopoietic lineage defects or defects of thymus stromal development or function. The latter includes athymia associated with DiGeorge syndrome (DGS), known as complete DGS (cDGS), and other (mono) genetic defects resulting in failure of thymic stromal development. While allogenic thymus transplantation has been shown to promote survival in around half of cases of cDGS, an otherwise lethal condition, the quality of immune reconstitution is poor with low numbers of naïve T-cells and a restricted T-cell repertoire, often resulting in various auto-immune complications. The present invention provides corrective therapies that involve providing such patients with thymic tissue to facilitate the development of their normal hematopoietic precursor cells into mature T-cells. Furthermore, the thymus, the primary lymphoid organ crucially required for generation of a functional T-cell repertoire, is the first organ to undergo age-related degeneration (thymic involution) during normal ageing. Thymic involution is a critical factor in the impaired capacity of adult patients to recover an adaptive immunity following a therapeutic immune depletion. Thus, development of improved thymus-based therapies for enhancing immune system function in patients is of broad interest.

Thymic epithelial cells (TECs) are critical effectors in the intrathymic microenvironments required for T-cell development. Two distinct TEC sub-lineages exist, located in the cortical and medullary compartments of the thymus. These cortical (c) and medullary (m) TECs mediate discrete aspects of T-cell development, and their segregation into distinct compartments is thought vital for accurate and efficient production of a self-restricted, self-tolerant T-cell repertoire. The cortex is involved in setting up the T lymphoid differentiation program, mainly be acting via the DLL4 (Delta Like Canonical Notch Ligand 4) Notch ligand on cortical epithelial cells. Notch signaling is required for inducing T cell development. In the medulla, among other processes, positive (for one's own HLA) and negative selection (against auto-antigens) occur. Despite their functional differences, cTECs and mTECs initially arise from a common TEC progenitor cell (thymus epithelial progenitor cell; TEPC), and the forkhead transcription factor FOXN1, expressed exclusively in thymic and cutaneous epithelia, is required at multiple stages for differentiation of both sub-lineages. Neonatal thymus transplantation can confer adaptive immunity to congenitally athymic patients, however its widespread use is limited by donor tissue supply and lack of any major histocompatibility matching; these limitations would be overcome if functional TECs could be generated or expanded in vitro.

Therefore, the invention provides a method wherein a nucleic acid molecule comprising a FOXN1 sequence is introduced in a safe harbour locus of a pluripotent cell or is randomly introduced in the genome.

In one aspect, the invention provides a method for producing a three dimensional thymic like structure, the method comprising: a) providing pluripotent cells from a recipient with a nucleic acid molecule comprising a FOXN1 sequence, and b) inducing said pluripotent cell to differentiate into thymic epithelium in a scaffold suitable for transplantation. In embodiments, step a) of the method is performed ex vivo. In embodiments, step b) of the method is performed ex vivo.

In one aspect, the invention provides a method for producing a three dimensional thymic like structure, the method comprising: a) providing pluripotent cell(s) from a recipient, and b) inducing said pluripotent cell(s) to differentiate into thymic epithelium in a scaffold suitable for transplantation,
wherein a nucleic acid molecule comprising a FOXN1 sequence is provided to the pluripotent cell(s) from a recipient of a) or to cells of a differentiation stage in b) selected from definitive endoderm cells, anterior foregut endoderm cells, and pharyngeal pouch endoderm cells. In embodiments, step a) of the method is performed ex vivo. In embodiments, step b) of the method is performed ex vivo.

For example, it may be possible when using retroviral, in particular lentiviral vectors for providing the vectors that the nucleic acid molecule comprising a FOXN1 sequence is provided during a differentiation stage observed in step b). In step b), pluripotent cell(s) are induced to differentiate into thymic epithelium. During differentiation into thymic epithelium, the pluripotent cell(s) differentiate to definitive endoderm (DE) cells, then to anterior foregut endoderm (AFE) cells, and subsequently to pharyngeal pouch endoderm (PPE) cells, followed by differentiation to TEPC.

Herein, a “three dimensional thymic like structure” is understood as a three-dimensional (3D) structure comprising one or more thymus epithelial cell (TEC) and/or thymus epithelial progenitor cell (TEPC), which is able to direct hematopoietic progenitor cells towards the T-lymphocyte lineage, in vivo and/or in vitro. In embodiments, the hematopoietic progenitor cells used herein consist of CD34⁺ hematopoietic stem cells.

“direct” towards the T cell lineage in vitro and/or in vivo” is understood as to promote the formation of cells in vitro and/or in vivo which exhibit at least one cell surface marker indicative for commitment of the hematopoietic progenitor cell along the T cell development trajectory. In humans, a cell of T cell-lineages or T cell lineage is understood as a cell selected from one of the following populations, in the order from early progenitor to mature T cells: 1) CD34+CD7+CD1a−, 2) CD7+CD5−CD4−CD8−CD3− population, 3) CD7+CD5+CD4−CD8−CD3− TCRαβ− population, 4) CD7+CD5+ CD4+ CD8−CD3− TCRαβ− immature single positive T cells, 5) CD7+CD5+ CD4+ CD8+ CD3− TCRαβ− double positive 6) CD7+CD5+CD4+CD8+CD3+ TCRαβ+ and 7) CD4+CD3+ TCRαβ+ or 8) CD8+ CD3+ TCRαβ+ mature single positive. For mice, this lineage consists of 1) CD44+ CD25− CD4−CD8−CD3− TCRαβ−, 2) CD44+ CD25+ CD4−CD8−CD3− TCRαβ−, 3) CD44− CD25+ CD4−CD8−CD3− TCRαβ− 4) CD44− CD25− CD4− CD8−CD3− TCRαβ−, 5) CD4−CD8+ CD3− TCRαβ− immature single positive 6) CD4+CD8+CD3+ TCRαβ+ double positive and 7) CD4+ CD3+ TCRαβ+ or CD8+CD3+ TCRαβ+ mature single positives.

Accordingly, the three dimensional thymic like structure provided herein, or the spheroid or organoid herein, is able to promote the formation of cells in vitro and/or in vivo which exhibit at least one cell surface marker indicative for commitment of the hematopoietic progenitor cell along the T cell development trajectory.

In embodiments, three dimensional thymic like structure provided herein, or the spheroid or organoid herein, promotes the formation of a CD34+CD7+ CD1a− population from hematopoietic progenitor cell. In embodiments, three dimensional thymic like structure provided herein, or the spheroid or organoid herein, promotes the formation of a CD7+CD5−CD4−CD8−CD3− population from hematopoietic progenitor cell. In embodiments, three dimensional thymic like structure provided herein, or the spheroid or organoid herein, promote the formation of a CD7+ CD5+CD4−CD8−CD3− TCRαβ− population from hematopoietic progenitor cell. In embodiments, a three dimensional thymic like structure provided herein, or the spheroid or organoid herein, promotes the formation of a CD7+CD5+ CD4+ CD8−CD3− TCRαβ− immature single positive T cells from hematopoietic progenitor cell. In embodiments, a three dimensional thymic like structure provided herein, or the spheroid or organoid herein, promote the formation of a CD7+CD5+ CD4+ CD8+ CD3− TCRαβ− double positive population from hematopoietic progenitor cell. In embodiments, a three dimensional thymic like structure provided herein, or the spheroid or organoid herein, promote the formation of a CD4−CD8+ CD3− TCRαβ− immature single positive population from hematopoietic progenitor cell. In embodiments, a three dimensional thymic like structure provided herein, or the spheroid or organoid herein, promotes the formation of a CD4+CD8+CD3+ TCRαβ+ double positive population from hematopoietic progenitor cell. In embodiments, a three dimensional thymic like structure provided herein, or the spheroid or organoid herein, promotes the formation of a CD4+ CD3+ TCRαβ+ or CD8+CD3+ TCRαβ+ mature single positives population from hematopoietic progenitor cell. A “thymic epithelium” is understood as a composition comprising TEPC and/or TEC. The term “three dimensional thymic like structure” is also referred to herein as “thymic spheroid”, “spheroid”, “organoid” or “thymic organoid” or artificial thymic organoid (ATO). The terms TEPC and TEC are intended to encompass iTEPCs and iTECs, respectively. The term “thymus organoid” or “thymus spheroid”, also described as “organoid” or “spheroid”, refers to an artificial thymus organ produced preferably in vitro that resembles the 3D thymic structure.

In some embodiments, the “thymus organoid”, “thymus spheroid”, “organoid” or “spheroid”, mimics thymus function, in particular by facilitating T cell or T cell lineage development in vitro and in vivo. In embodiments, the thymus organoid is of human origin. In embodiments, the cells of the thymus organoid are of human origin. In embodiments, the thymus organoid comprises autologous cells or HLA-matched cells or comprises cells derived from autologous cells or HLA-matched cells.

A “scaffold suitable for transplantation” is understood as a structural component, comprising supporting cells and/or an acellular component such as a material mimicking extracellular matrix, on which TEPCs and/or TECs can be cultured. The acellular component is also referred to as “supporting matrix” herein. In embodiments, the scaffold comprises a supporting matrix. In embodiments, a supporting matrix comprises hyaluronic acid or a LDEV-Free Reduced Growth Factor Basement Membrane Matrix (such as Geltrex™), or a mixture thereof. In other embodiments, the scaffold comprises mesenchymal stromal cells (MSC). In such embodiments, the scaffold may or may not further comprise a supporting matrix.

In embodiments, said scaffold comprises cells derived from the recipient and/or HLA-matched with such recipient. Such cells in the scaffold are also referred to as “supporting cells” herein. For example, fibroblasts or mesenchymal stromal cells may be used. Accordingly, the “scaffold suitable for transplantation” comprises a supporting matrix and/or supportive cell. In embodiments, the “scaffold suitable for transplantation” comprises a supporting matrix. In embodiments, the “scaffold suitable for transplantation” comprises a supporting matrix or supportive cells. In embodiments, the “scaffold suitable for transplantation” comprises supportive cells. In embodiments, the “scaffold suitable for transplantation” comprises a supporting matrix and supportive cells.

The method comprises in one step providing pluripotent cells from a recipient with a nucleic acid molecule comprising a FOXN1 sequence.

The term “nucleic acid molecule” or “nucleic acid” is herein understood to mean RNA, PNA and/or DNA, wherein DNA is preferred.

The nucleic acid may be provided to pluripotent cells from a recipient in the form of a vector. In here, to distinguish from viral vectors (gene delivery vehicles) vectors are sometimes referred to as nucleotide vectors. In the following, referencing to a viral vector is also understood as gene delivery vehicles. The vector is preferably a molecule of circular double-helical DNA. In embodiments, the vector is of lentiviral origin. The purpose of the vector is to transfer genetic information to a target cell, preferably to a pluripotent cell or its differentiated progeny. In embodiments, the pluripotent cell is from the recipient of the spheroid or organoid prepared. Methods for providing pluripotent cells from a recipient with a nucleic acid molecule comprising a FOXN1 sequence are known in the art and include viral transduction and non-viral transduction (transfection) methods, in particular lentiviral transduction methods.

The term “FOXN1 sequence” is used to indicate a nucleotide sequence encoding for the Forkhead Box N1 transcription factor. Forkhead Box N1 transcription factor is required for development of the thymus epithelium. The naturally occurring FOXN1 gene sequence is for example disclosed in the NIH Gene database with Gene ID: 8456 (as updated on 23 Nov. 2023). The human protein sequence is disclosed e.g. as NCBI Reference Sequence NP_003584.2 (version of Feb. 19, 2024). The consensus coding sequence is disclosed in NCBI CCDS ID CCDS11232.1. In embodiments, the FOXN1 sequence is encoding a human FOXN1 protein.

In embodiments, the FOXN1 sequence is codon optimized for human codon usage. In embodiments, said codon optimization comprises removal of at least one CpG island and/or at least one cryptic splice site. In embodiments, said codon optimization comprises removal of at least one CpG island and at least one cryptic splice site The codon optimized sequence is optimized for codon usage that best fits with human tRNAs (Transfer Ribonucleic Acids), which improves expression.

In embodiments, the FOXN1 encoding sequence is under the control of an inducible promoter. The nucleic acid molecule further preferably comprises an inducible promoter that controls the expression of the FOXN1 sequence. In embodiments, the nucleic acid comprising the inducible promoter is operably linked to the FOXN1 sequence, to allow transcription under the control of the inducible promotor.

The term “inducible promoter” is understood by a person skilled in the art as a genetic element that can control the binding of RNA polymerase and transcription factors and thereby controls the transcription of a target gene.

Many inducible promoter systems are known, including the tetracycline-inducible expression systems, wherein tetracycline or its analogs are used as inducer.

In embodiments, the inducible promoter is a tetracycline-inducible promoter (TET on or TET off promoter), an estrogen inducible promoter (e.g. a promoter inducible by estradiol or analogues thereof or by tamoxifen, a cumate-controlled promotor (e.g. VP16), a FKBP12 and/or cyclophilin inducible promoter, a FRAP (protein-protein interaction induced by small molecule) promoter, or a metallothionein promoter. Such inducible promoter systems are known to a skilled person.

In certain embodiments, the nucleic acid molecule further preferably comprises DNA sequences that are homologous to a safe harbour locus of the target cell which promote introduction of the FOXN1 sequence. In other embodiments, the nucleic acid molecule is for random integration into the genome of the target cell.

In one aspect, a nucleic acid molecule comprising a FOXN1 sequence is provided, wherein the nucleic acid further comprises nucleic acid sequences for introducing said FOXN1 sequence in a pluripotent cell.

In embodiments, the nucleic acid further comprises nucleic acid sequences for introducing said FOXN1 sequence in a safe harbour locus of a pluripotent cell.

In such embodiments, the FOXN1 is integrated into the genome. In one aspect, a vector is provided comprising a nucleic acid molecule provided herein. In embodiments, the nucleic acid sequences for introducing a FOXN1 sequence in a safe harbour locus of a pluripotent cell comprise homologous sequences to a safe harbour locus. In embodiments, the safe harbour locus is AAVS1, CCR5, or humanRosa26. For example, CRISPR/Cas-mediated integration may be used for integration into a safe harbour locus.

In said embodiments, said TEPC comprising a FOXN1 encoding sequence comprises said FOXN1 encoding sequence integrated in its DNA in a safe harbour locus.

In other embodiments, the FOXN1 encoding sequence is randomly integrated into the target cell. For example, retroviral transductions, in particular lentiviral transduction is used for random integration. In embodiments, the nucleic acid does not further comprise nucleic acid sequences for introducing said FOXN1 sequence in a safe harbour locus of a pluripotent cell.

In other embodiments, the vector remains episomal upon introduction to a pluripotent cell.

For example, AAV vectors may be used as vector which remains episomal upon introduction to a pluripotent cell.

In other embodiments, the vector is integrated upon introduction to a pluripotent cell.

In aspects, a viral vector comprising a vector provided herein and a viral capsid is provided.

In embodiments, a lentiviral vector of lentiviral origin.

Retroviral vectors, including lentiviral vectors, are capable of integrating nucleic acids into host cell genomes.

The viral vector, such as the retroviral vector, including lentiviral vectors, may be introduced into the pluripotent cell(s). In such embodiments, the viral vector is introduced prior to differentiation stage. In other embodiments, the viral vector is introduced at one of the differentiation stages of definitive endoderm cells, anterior foregut endoderm cells, or pharyngeal pouch endoderm cells.

In embodiments, the viral vector is introduced into the pluripotent cell(s). In other embodiments, the viral vector, such as the retroviral vector, including lentiviral vectors, is introduced into the cell(s) of one of the differentiation stages, of definitive endoderm cells, anterior foregut endoderm cells, or pharyngeal pouch endoderm cells.

In embodiments, the viral vector, such as the retroviral vector, including lentiviral vectors, is introduced into the cell(s) of differentiation stage of definitive endoderm cells. In embodiments, the viral vector, such as the retroviral vector, including lentiviral vectors, is introduced into the cell(s) of differentiation stage of anterior foregut endoderm cells. In embodiments, the viral vector, such as the retroviral vector, including lentiviral vectors, is introduced into the cell(s) of differentiation stage of pharyngeal pouch endoderm cells.

A retroviral vector, including lentiviral vectors, may be introduced into pluripotent cell(s), in cells of differentiation stages of definitive endoderm cells, anterior foregut endoderm cells, or pharyngeal pouch endoderm cells.

For other viral vectors, such as for introduction into the safe harbour locus of a target cells, the viral vector is preferably introduced into the pluripotent cell(s).

The term “safe harbour locus” indicates genomic locations where genes or genetic elements can be introduced without disrupting the expression or regulation of adjacent genes. Preferably, the safe harbour locus includes, but is not limited to, the Adeno-Associated Virus Integration Site 1 (AAVS1) locus, CCR5, human Rosa26, and other loci.

In one aspect of the present invention, viral vectors are provided. The viral vectors include an inducible promoter operably linked to a polynucleotide encoding a FOXN1 polypeptide. The general approach in certain aspects of the present invention is to provide a pluripotent cell with an inducible expression construct encoding a FOXN1 polypeptide, thereby permitting the expression of the FOXN1 polypeptide in the cell upon induction of the promoter at an appropriate tie point during differentiation to a TEPC. Following delivery of the expression construct, the FOXN1 polypeptide encoded by the expression construct is synthesized by the transcriptional and translational machinery of the cell.

As used herein, an “expression construct encoding FOXN1 polypeptide” refers to a promoter operably connected to a polynucleotide encoding a FOXN1 polypeptide. In embodiments, the promoter is inducible.

In embodiments of the invention, the expression construct encoding the FOXN1 polypeptide is stably integrated into the genome of the cell. In other embodiments, the expression construct encoding the FOXN1 polypeptide is stably or transiently maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and/or where in the cell the nucleic acid remains is dependent on the type of vector employed.

The viral vector may be a virus particle or may be encoded on a DNA plasmid. In some embodiments where the viral vector is a virus particle, for example a lentivirus viral particle, the virus particle may include a VSV-G envelope protein or other suitable env gene encoded proteins. The capacity of certain viral vectors to efficiently infect or enter cells, to integrate into a host cell genome and stably express viral genes, have led to the development and application of a number of different viral vector systems. For example, adenovirus, herpes-simplex virus, retrovirus and adeno-associated virus vectors may be used. Suitable viral vectors that may be used include retroviral vectors, adeno-associated viral (AAV) vectors, adenoviral vectors, or herpes-simplex vectors. Retroviral vectors may include, for example, lentiviral vectors.

Retroviruses or retroviral vectors are RNA viruses comprising an RNA genome. When a host cell is infected by a retrovirus, the genomic RNA is reverse transcribed into a DNA intermediate which is integrated into the chromosomal DNA of infected cells. This integrated DNA intermediate is referred to as a provirus. A particular advantage of retroviruses is that they can stably infect dividing cells with a gene of interest (e.g., a therapeutic gene) by integrating into the host DNA, without expressing immunogenic viral proteins. Theoretically, the integrated retroviral vector will be maintained for the life of the infected host cell, expressing the gene of interest.

Lentiviral vectors are a type of retrovirus that can infect both dividing and nondividing cells. Lentiviruses can be used to provide highly effective gene therapy as lentiviruses can enable expression of a gene of interest in their target cells and their progeny.

Illustrative lentiviruses include, but are not limited to: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Retroviral vectors and more particularly lentiviral vectors may be used in some embodiments. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus.

The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging. In particular embodiments, the terms “lentiviral vector” and “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

At each end of the provirus are structures called “long terminal repeats” or “LTRs”. The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR comprises U3, R, and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site). As used herein, the term “packaging signal” or “packaging sequence” refers to sequences located within the retroviral genome which are required for insertion of the viral RNA into the viral capsid or particle. Several retroviral vectors use the minimal packaging signal (also referred to as the psi [W] sequence) needed for encapsidation of the viral genome. Thus, as used herein, the terms “packaging sequence”, “packaging signal”, “psi” and the symbol “W” are used in reference to the non-coding sequence required for encapsidation of retroviral RNA strands during viral particle formation. In various embodiments, vectors comprise modified 5′ LTR and/or 3′ LTRs. Either or both of the LTRs may comprise one or more modifications including, but not limited to, one or more deletions, insertions or substitutions. Modifications of the 3′ LTR are often made to improve the safety of lentiviral or retroviral systems by rendering viruses' replication defective. As used herein, the term “replication-defective” refers to virus that is not capable of complete, effective replication such that infective virions are not produced (e.g., replication-defective lentiviral progeny). The term “replication-competent” refers to wildtype virus or mutant virus that is capable of replication, such that viral replication of the virus is capable of producing infective virions (e.g., replication-competent lentiviral progeny).

Adeno-associated virus (AAV), a member of the parvovirus family, is a human virus that is increasingly being used for gene delivery approaches. AAV can infect a wide range of host cells, including non dividing cells. AAV has not been associated with any human or animal disease and does not appear to alter the biological properties of the host cell. For example, it is estimated that 80-85% of the human population has been exposed to AAV. AAV is stable at a wide range of physical and chemical conditions which lends itself to production, storage and transportation requirements.

The AAV genome is a linear, single-stranded DNA molecule containing 4681 nucleotides. The AAV genome generally comprises an internal non-repeating genome flanked on each end by inverted terminal repeats (ITRs) of approximately 145 bp in length. The ITRs have multiple functions, including origins of DNA replication, and as packaging signals for the viral genome. AAV ITRs may be derived from any of several AAV serotypes, including AAV1, AAV2, AAV3, AAV4, AAV5, AAV6, AAV7, AAV8, AAV9, AAV10, AAV11, avian AAVs, bovine AAVs etc. The 5′ and 3′ ITRs of the AAV viral vectors disclosed herein may be derived from any of these AAV serotypes.

The internal non-repeated portion of the AAV genome includes two large open reading frames, known as the AAV replication (rep) and capsid (cap) genes. The rep and cap genes code for viral proteins that allow the virus to replicate and package the viral genome into a virion.

AAV is a helper-dependent virus requiring co-infection with a helper virus (e.g., adenovirus, herpesvirus or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. Although AAV can infect cells from different species, the helper virus must be of the same species as the host cell (e.g., human AAV will replicate in canine cells co-infected with a canine adenovirus).

Suitable AAV vectors are known in the art. For example, suitable AAV vectors include AAV2/5, demonstrated in “AAV2/5-mediated gene therapy in iPSC-derived retinal pigment epithelium of a choroideremia patient” (Cereso et. al. Mol Ther Methods Clin Dev. 2014).

Herpes simplex virus (HSV) type I and type II contain a double-stranded, linear DNA genome of approximately 150 kb, encoding 70-80 genes. Wild type HSV are able to infect cells lytically and to establish latency in certain cell types (e.g., neurons). Similar to adenovirus, HSV also can infect a variety of cell types. For use in therapeutic gene delivery, HSV must be rendered replication-defective. Protocols for generating replication-defective HSV helper virus-free cell lines have been described (U.S. Pat. Nos. 5,879,934; 5,851,826).

A method according to the invention for producing a thymus epithelial progenitor cell (TEPC) from a pluripotent cell comprises transducing said pluripotent cell with a nucleic acid molecule comprising FOXN1 and contacting said transduced cell with a cocktail of growth factors. The cocktail of growth factors represents a differentiation protocol. Thereby, the pluripotent cell differentiates into TEPC via the intermediate stages of definitive endoderm cells, anterior foregut endoderm cells, and pharyngeal pouch endoderm cells. Such differentiation protocols are for example described in the Examples. The differentiation protocol may comprise the stepwise and timed modulation of key developmental pathways including the wnt, SHH, TGF, BMP and/or retinoic acid signaling pathways. Such differentiation protocols are well-known in the art and are for example provided in the examples. Further suitable differentiation protocols known in the art include (Chhatta et. al. Journal of Allergy and Clinical Immunology (2019), Parent et. al. Cell Stem Cell (2013), Ramos et. al. Stem Cell Reports (2023), Gras-Peña et. al. Journal of Allergy and Clinical Immunology (2022), Otsuka et. al. Scientific Reports (2020)).

In aspects, a method for producing a thymic epithelial progenitor cell (TEPC) from a pluripotent cell is provided comprising the steps: providing said pluripotent cell with a nucleotide vector provided herein, thereby obtaining a transduced cell, and subjecting said transduced cell to a differentiation protocol, optionally wherein the differentiation protocol comprises the stepwise and timed modulation of key developmental pathways including the wnt, SHH, TGF, BMP and/or retinoic acid signaling pathways.

In aspects, a method for producing a TEPC from a pluripotent cell is provided comprising the steps: providing a pluripotent cell and subjecting the pluripotent cell to a differentiation protocol to a TEPC, wherein a cell is infected with a viral vector provided herein, and wherein the cell is selected from the pluripotent cell and a cell of a differentiation stage to TEPC, in particular wherein the differentiation stage cell is selected from a definitive endoderm cell, anterior foregut endoderm cell, and pharyngeal pouch endoderm cell,
optionally wherein the differentiation protocol comprises stepwise and timed modulation of key developmental pathways including the Wnt, SHH, TGFB, BMP and/or retinoic acid signaling pathways.

In aspects, a method for producing a TEPC from a pluripotent cell is provided comprising the steps: infecting a pluripotent cell with a viral vector provided herein, thereby obtaining a transduced cell, and subjecting said transduced cell to a differentiation protocol,
optionally wherein the differentiation protocol comprises the stepwise and timed modulation of key developmental pathways including the wnt, SHH, TGF, BMP and/or retinoic acid signaling pathways.

In embodiments, the methods for producing a TEPC are ex vivo or in vitro methods.

In embodiments of methods herein, the pluripotent cell differentiates into TEPC via the intermediate stages of definitive endoderm cells, anterior foregut endoderm cells, and pharyngeal pouch endoderm cells and/or wherein the pluripotent cell differentiates into definitive endoderm cells and the differentiation of definitive endoderm to anterior foregut endoderm is induced when the percentage of SP CD117+ is at least 30% and the percentage of DP CD117+CD184+ cells is at most 70%.

In embodiments of methods herein, the pluripotent cell differentiates into TEPC via the intermediate stages of definitive endoderm cells, anterior foregut endoderm cells, and pharyngeal pouch endoderm cells.

In embodiments, the differentiation of pluripotent cell(s) to definitive endoderm is induced by exposing the cells to Activin A and optionally exposing cells to a Wnt-activator, optionally with Wnt3a and/or CHIR99021.

Methods to induce definitive endoderm are well known in the state of the art, and are for example described in the examples and further suitable protocols known in the art include d'Amour et al. (Efficient differentiation of human embryonic stem cells to definitive endoderm. Nature biotechnology 23.12 (2005): 1534-1541) or reviewed in Wang et al. (“Improvement of cell survival during human pluripotent stem cell definitive endoderm differentiation.” Stem Cells and Development 24.21 (2015): 2536-2546). In embodiments of methods herein, the pluripotent cell differentiates into a definitive endoderm cell, and the differentiation of definitive endoderm to anterior foregut endoderm is induced when the percentage of SP CD117+ is at least 30% and the percentage of DP CD117+CD184+ cells is at most 70%.

In embodiments, the differentiation of definitive endoderm to anterior foregut endoderm is induced by exposing the cells to dual TGFβ/BMP4 inhibition.

In embodiments, the differentiation of definitive endoderm to anterior foregut endoderm is induced by exposing the cells to dual TGFβ/BMP inhibition.

In some embodiments, dual TGFβ/BMP inhibition is by SB431542, noggin or a combination thereof. In some embodiments, dual TGFβ/BMP inhibition is by a combination of SB431542 and noggin.

In embodiments, FOXN1 expression is induced at the pharyngeal pouch endoderm stage. In embodiments, FOXN1 expression is induced at the definitive endoderm stage. In embodiments, FOXN1 expression is induced at the anterior foregut endoderm stage.

In embodiments, the cells of the thymus organoid are of human origin.

In embodiments herein, the safe harbour locus is AAVS1, CCR5, or humanRosa26.

In embodiments herein, the safe harbour locus is AAVS1, CCR5, humanRosa26 or SHS231.

In embodiments, the FOXN1 encoding sequence is under the control of an inducible promoter.

In embodiments, the inducible promoter is a tetracycline-inducible promoter (TET on or TET off promoter), an estrogen inducible promoter (e.g. a promoter inducible by estradiol or analogues thereof or by tamoxifen, a cumate-controlled promotor (e.g. VP16), a FKBP12 and/or cyclophilin inducible promoter, a FRAP (protein-protein interaction induced by small molecule) promoter, or a metallothionein promoter.

The term “pluripotent cell” refers to any type of cell that has the capacity to self-renew by dividing and to differentiate into a plurality of cell lineages. In embodiments, a pluripotent cell is understood as cell which is able to differentiate into ectoderm, endoderm and mesoderm lineage.

Preferably, the pluripotent cell is an induced pluripotent stem cell (iPSC).

The term, “induced pluripotent stem cells”, refers to a type of pluripotent cell that is produced from differentiated adult cells that have been induced or changed, i.e., reprogrammed into cells capable of differentiating into various tissues. Methods for providing a pluripotent cell, and methods for providing an iPSC are well known in the art and are, for example, described in Kim et. al. Nat Rev Mol Cell Biol 21 (2020). By providing methods of introducing the FOXN1 sequence under control of an inducible promoter in a pluripotent cell, either in a safe harbour locus of a pluripotent cell or randomly integrated, thereby providing pluripotent cells from a recipient with a nucleic acid molecule comprising a FOXN1 sequence, the inventors have established improved means and methods for differentiation of pluripotent cells towards thymic epithelium, from which a de novo thymus can be generated. The de novo thymus is provided herewith as “three dimensional thymic like structure”, organoid, thymic organoid, spheroid or thymic spheroid.

In aspects, a TEPC comprising a FOXN1 encoding sequence integrated in its DNA in a safe harbour locus is provided.

In aspects, a TEPC comprising a FOXN1 encoding sequence integrated in its DNA is provided.

In embodiments, said TEPC comprising a FOXN1 encoding sequence comprises said FOXN1 encoding sequence randomly integrated in its DNA.

The terms “pluripotent cell” and “nucleic acid molecule” or “nucleic acid” are defined as set out above. The term “TEPC” is used to describe cells that are progenitor cells of thymic epithelial cells that comprise the thymus. Methods for determining a TEPC are known in the art. For example, a TEPC can be identified as being EpCAM+ Podoplanin+ in the post-natal human thymus. From a developmental point of view, TEPCs are characterized as arising from a definitive endoderm (DE) population and the TEPC of the present invention are further expressing FOXN1.

Preferably, the TEPCs are prepared starting from induced pluripotent stem cells, which are differentiated into definitive endoderm (DE), anterior foregut endoderm (AFE), pharyngeal pouch endoderm (PPE) and finally to TEPCs. Definitive endoderm can be characterized as a SOX17+ (OCT4−) population or using the commonly-used surface markers CD117+CD184+. AFE cells are typically FOXA2+ cells arising from DE cells. PPE cells can be determined to express HOXA3. TEPC as provided herein express FOXN1. Typically, TEPCs express cytokeratins (e.g. Cytokeratin-8), EpCAM, DLL4, and/or Podoplanin.

The term “transduction” is used as understood by a person skilled in the art as a process by which foreign DNA, preferably the nucleic acid molecule, is introduced into a cell by any means, preferably by a viral vector, which typically comprises a recombinant transgene and a viral capsid, such as derived from a lentiviral vector. The term “recombinant lentiviral vector” is defined as a vehicle for gene delivery that was originally derived from a lentivirus, preferably the human immunodeficiency virus type-1 (HIV-1) lentivirus.

By providing methods of producing a TEPC expressing FOXN1 from a pluripotent cell, the inventors have established improved means and methods for generating an artificial thymus that can be used for treatment of various pathologies.

The invention further provides a spheroid or organoid that comprises at least one TEPC and/or at least one TEC. The invention further provides a spheroid or organoid that comprises at least one TEPC and at least one TEC.

The term “TEPC” is defined as set out above. Methods for determining a TEPC are known in the art. For example, a TEPC can be identified as being from endodermic origin (definitive endoderm) and expressing the FOXN1 gene.

A TEC can be induced from said TEPC by contacting the TEPC with a hematopoietic stem cell or a common lymphoid progenitor or a T-cell progenitor under conditions allowing for said induction or by further ex vivo or in vivo maturation.

Such conditions include, for example co-cultures of TEPC with human CD34+ cells as aggregates on an air-liquid interface.

Air-liquid interface culture methods are known in the field and can be described as followed: a porous membrane is used that allows faster diffusion of nutrients via the liquid phase (under) and high oxygenation via the air phase (above).

The thymus organoid or spheroid has the capacity to induce T-cell development in vitro and/or in vivo.

The thymus organoid or spheroid is of mammalian origin, of murine origin or preferably of human origin. In embodiments, the cells of the thymus organoid are of human origin.

The spheroid or organoid can comprise supporting cells.

In embodiments of a method for inducing a thymic epithelial cell (TEC) from a TEPC, the method comprises contacting said TEPC with an autologous hematopoietic stem cell or autologous hematopoietic progenitor cell or an autologous common lymphoid progenitor or an autologous T-cell progenitor under conditions allowing for said induction.

In embodiments of a method for inducing a thymic epithelial cell (TEC) from a TEPC, the method comprises contacting said TEPC with an autologous hematopoietic stem cell or an autologous common lymphoid progenitor or an autologous T-cell progenitor under conditions allowing for said induction, optionally wherein said contacting comprises co-culturing said TEPC with an autologous hematopoietic stem cell or an autologous common lymphoid progenitor or an autologous T-cell progenitor.

In embodiments, said contacting or co-culturing is in a spheroid or organoid or artificial thymic organoid (ATO).

Provided is a TEC obtainable by a method for inducing a thymic epithelial progenitor cell (TEC) from a TEPC.

In an aspect, a method of producing at least one T cell or T lineage cell is provided, wherein the method comprises the steps of: a) providing a spheroid or organoid as provided herein comprising at least one thymus epithelial cell (TEC) and/or thymus epithelial progenitor cell (TEPC), b) contacting the spheroid or organoid of a) with at least one hematopoietic progenitor cell or T lineage cell, thereby producing at least one T cell or T lineage cell.

In embodiments, the method is an ex vivo method. In embodiments, the method is an in vivo method. In an embodiment, the cell in b) is at least one hematopoietic progenitor cell. In an embodiment, the at least one hematopoietic progenitor cell is a CD34+ cell. In an embodiment, the at least one hematopoietic progenitor cell is an autologous cell. In an embodiment, the at least one T cell or T lineage cell is one or more of the following cells of 1) CD34+CD7+ CD1a−, 2) CD7+CD5−CD4−CD8−CD3− population, 3) CD7+ CD5+CD4−CD8−CD3−TCRαβ− population, 4) CD7+CD5+ CD4+ CD8−CD3− TCRαβ− immature single positive T cells, 5) CD7+CD5+ CD4+ CD8+ CD3− TCRαβ− double positive 6) CD7+CD5+ CD4+ CD8+ CD3+TCRαβ+, 7) CD4+CD3+ TCRαβ+ or 8) CD8+ CD3+ TCRαβ+ mature single positive. For mice, this lineage consists of 1) CD44+ CD25− CD4−CD8−CD3− TCRαβ−, 2) CD44+ CD25+ CD4−CD8−CD3− TCRαβ−, 3) CD44−CD25+CD4−CD8−CD3− TCRαβ− 4) CD44− CD25− CD4−CD8−CD3− TCRαβ−, 5) CD4−CD8+ CD3− TCRαβ− immature single positive 6) CD4+CD8+CD3+ TCRαβ+ double positive, 7) CD4+ CD3+ TCRαβ+ or 8) CD8+CD3+ TCRαβ+ mature single positives. In some embodiments, the cells in b) are T lineage cells. In such embodiments, the T lineage cells in b) are different from the T lineage cells produced by the method. In particular, the T lineage cells in b) are less mature and/or more progenitor than the T cell progenitor produced. T cell lineage cells described above are listed from more progenitor to more mature by their marker profile. For example, the T cell lineage in b) may be a T cell lineage cell recited above which exhibits a CD7+CD5+ marker profile, such as CD7+CD5−CD4−CD8−CD3− or CD7+ CD5+CD4−CD8−CD3−TCRαβ−, and the T cell lineage cell produced may be a CD4+ single positive cell, such as CD4+ CD3+ TCRαβ+ SP. In embodiments, said contacting comprises co-culturing. In embodiments, said contacting comprises co-culturing for at least 5 or more days, such as for 1, 2, 3, 4, 5, 6, 7, 8, 9 or 10 weeks.

In a further aspect, a thymus spheroid or organoid comprising at least one TEPC provided herein and/or at least one TEC provided herein, is provided.

In a further aspect, a spheroid or organoid herein is provided wherein the spheroid or organoid further comprising supporting cells.

The term “supporting cells” is defined as a set of cells that give structural integrity to the spheroid or organoid, and may comprise of mesenchymal stromal cells, fibroblasts, or other. “mesenchymal stromal cells” are defined as spindle shaped plastic-adherent cells isolated from bone marrow, adipose, and other tissue sources, with multipotent differentiation capacity. “fibroblasts” are a type of cell that contribute to the formation of connective tissue, a fibrous cellular material that supports and connects other tissues or organs in the body. Instead of the supporting cells, a “supporting matrix” could be used to introduce structural integrity to the spheroid or organoid. The “supporting matrix” can be based on materials such as (GMP-grade) Collagen type 1, hydrogels based on fibronectin, laminins, and other proteoglycans. Alginate, hyaluronic acid-based gels or commercially available matrixes (Like Hystem). In embodiments, the supporting matrix comprises 3D printed materials. Suitable 3D or three-dimensional printed materials and methods are known in the art as e.g. disclosed in Moroni et al. Nature Reviews Materials (2018) and Abaci et al. Advanced healthcare materials (2020).

The supporting cells preferably share their origin with TEPCs and or TECs that comprise the spheroid or organoid. Shared origin here is described as arising from the same iPSC line or from other autologous sources such as adipose tissues or bone marrow (for MSC). In embodiments, shared origin here is described as arising from the same individual to be treated. Accordingly, in embodiments, the iPSCs are from autologous cells. Additionally, the spheroid or organoid, comprising supporting cells or not, can comprise a supporting matrix.

The spheroid or organoid can be used in cell therapy, such as for the treatment of an immune deficiency, DiGeorge syndrome, FOXN1 deficiency, or a lack of a functional thymus, for example as a result of chemotherapy, hematopoietic stem cell transplantation, thymectomy, thymus involution. The amount (or number) of spheroids or organoids administered to the patient may be determined by the physician and depends on the patient to be treated in need of the treatment. For example, amounts of spheroids or organoids are typically in the range of between 1 and 100,000. For example, 10 to 100,000, 100 to 100,000, 10 to 1,000, 100 to 10,000, or 500 to 50,000 spheroids or organoids, or any subrange thereof, may be administered. A spheroid or organoid herein may typically comprise between about 20,000 and 2.000,000,000 TEC and/or TEPC. For example, a spheroid or organoid herein may comprise between about 50,000 and 1,000,000,000, between about 100,000 and 100,000,000, between about 1,000,000 and 100,000,000, between about 1,000,000 and 10,000,000 TEC and/or TEPC any subrange thereof. The size of the spheroid or organoid may vary, and the diameter may be in the range of between 0.01 mm and 1 cm.

For example, the diameter may be in the range of between 0.01 mm and 0.1 cm, 0.05 mm to 5 mm, 0.1 mm to 0.5 mm, or 0.05 mm to 0.5 cm.

Thereby, the spheroids or organoids are useful for cell therapy. The term “cell therapy”, as understood by a person skilled in the art, refers to placing new, healthy cells into the body to replace diseased, damaged, or missing cells. The spheroid(s) or organoid(s) can be implanted in skeletal muscle, such as a quadriceps muscle. In embodiments, said muscle shares its origin with said spheroid or organoid. By providing methods of producing thymus spheroid or organoid, the inventors have established improved means and methods for therapy of patients that lack a functional thymus.

In embodiments, the thymus spheroid or organoid has a medulla-like and/or cortex-like three-dimensional structure (or 3D structure). In embodiments, the thymus spheroid or organoid having a medulla-like and/or cortex-like three-dimensional structure comprise cell dense (cortex) and lighter (medulla) areas. For example, the thymus spheroid or organoid comprising medulla-like and/or cortex-like three-dimensional structure comprises TEC which are cortical-like. In embodiments, the TECs are co-expressing CKT8, EpCAM and Podoplanin, preferably together in a cluster or in lobes of the thymus spheroid or organoid.

In aspects, provided herein is a spheroid or organoid provided herein for use in cell therapy.

In aspects, provided herein is a spheroid or organoid provided herein for use in the treatment of congenital and/or acquired diseases.

In aspects, provided herein is a spheroid or organoid provided herein for use in the treatment of an immune deficiency.

In aspects, provided herein is a spheroid or organoid provided herein for use in the treatment of a lack of a functional thymus.

In aspects, provided herein is a spheroid or organoid provided herein for use in the treatment of DiGeorge syndrome.

In aspects, provided herein is a spheroid or organoid provided herein for use in the treatment of FOXN1 deficiency.

In aspects, provided herein is a spheroid or organoid provided herein for use in the treatment of a lack of a fully functional immune repertoire.

In aspects, provided herein is a spheroid or organoid provided herein for use in the treatment of a lack of a fully functional immune repertoire due to a viral or bacterial infection.

In aspects, provided herein is a spheroid or organoid provided herein for use in the treatment of a lack of a fully functional immune repertoire as a result of chemotherapy.

In aspects, provided herein is a spheroid or organoid provided herein for use in the treatment of CHARGE syndrome.

In aspects, provided herein is a spheroid or organoid provided herein for use in the treatment of Otofaciocervical Syndrome.

In aspects, provided herein is a spheroid or organoid provided herein for use in the treatment of a lack of a fully functional immune repertoire as a result of hematopoietic stem cell transplantation.

In certain embodiments, the lack of a fully functional immune repertoire as a result of hematopoietic stem cell transplantation (HSCT) is due to myeloablative conditioning and/or chemotherapy as part of the HSCT treatment regime.

In aspects, provided herein is a spheroid or organoid provided herein for use in the treatment of a lack of a functional thymus as a result of thymectomy.

In aspects, provided herein is a spheroid or organoid provided herein for use in the treatment of a lack of a functional thymus as a result of thymus involution.

In embodiments herein, the spheroid or organoid is implanted in skeletal muscle.

In embodiments herein, the skeletal muscle is a quadriceps muscle.

In embodiments herein, said skeletal muscle shares its origin with said spheroid or organoid.

Provided is a method of cell therapy treatment of a patient in need thereof, the method comprising administering to said patient a therapeutically effective amount of at least one spheroid or organoid provided herein.

Provided is a method of treatment of congenital and/or acquired diseases of a patient in need thereof, the method comprising administering to said patient a therapeutically effective amount of at least one spheroid or organoid provided herein.

Provided is a method of treatment of an immune deficiency of a patient in need thereof, the method comprising administering to said patient a therapeutically effective amount of at least one spheroid or organoid provided herein.

Provided is a method of treatment of lack of a functional thymus in a patient in need thereof, the method comprising administering to said patient a therapeutically effective amount of at least one spheroid or organoid provided herein.

Provided is a method of treatment of DiGeorge syndrome in a patient in need thereof, the method comprising administering to said patient a therapeutically effective amount of at least one spheroid or organoid provided herein.

Provided is a method of treatment of CHARGE syndrome in a patient in need thereof, the method comprising administering to said patient a therapeutically effective amount of at least one spheroid or organoid provided herein.

Provided is a method of treatment of Otofaciocervical Syndrome in a patient in need thereof, the method comprising administering to said patient a therapeutically effective amount of at least one spheroid or organoid provided herein. Provided is a method of treatment of FOXN1 deficiency in a patient in need thereof, the method comprising administering to said patient a therapeutically effective amount of at least one spheroid or organoid provided herein.

Provided is a method of treatment of lack of a fully functional immune repertoire in a patient in need thereof, the method comprising administering to said patient a therapeutically effective amount of at least one spheroid or organoid provided herein.

Provided is a method of treatment of lack of a fully functional immune repertoire due to a viral or bacterial infection in a patient in need thereof, the method comprising administering to said patient a therapeutically effective amount of at least one spheroid or organoid provided herein. Provided is a method of treatment of lack of a fully functional immune repertoire as a result of chemotherapy in a patient in need thereof, the method comprising administering to said patient a therapeutically effective amount of at least one spheroid or organoid provided herein.

Provided is a method of treatment of lack of a fully functional immune repertoire as a result of hematopoietic stem cell transplantation in a patient in need thereof, the method comprising administering to said patient a therapeutically effective amount of at least one spheroid or organoid provided herein.

Provided is a method of treatment of lack of a functional thymus as a result of thymectomy in a patient in need thereof, the method comprising administering to said patient a therapeutically effective amount of at least one spheroid or organoid provided herein.

Provided is a method of treatment of lack of a functional thymus as a result of thymus involution in a patient in need thereof, the method comprising administering to said patient a therapeutically effective amount of at least one spheroid or organoid provided herein.

In embodiments of the methods and uses herein, the spheroid or organoid is implanted in skeletal muscle. In embodiments herein, the skeletal muscle is a quadriceps muscle. In embodiments herein, said skeletal muscle shares its origin with said spheroid or organoid. In embodiments, the skeletal muscle is syngeneic to the cells in the spheroid or organoid. For example, the cells in the spheroid or organoid are autologous to the patient.

### EXAMPLES

### Materials

Cell Culture and Differentiation of iPSCs

### MS5-hDLL4 & MS5-hDLL1

MS5-hDLL4 (MS5 (mouse stromal cell line) cells transduced with hDLL4) and MS5-hDLL1 (MS5 (mouse stromal cell line) cells transduced with hDLL1) (Merck, Sigma-Aldrich) cells were cultured in DMEM-high glucose/10% FCS/1% PenStrep and passaged once to twice a week, when 80% confluency was reached. For passaging, cells were washed once with PBS and incubated with 0.05% Trypsin-EDTA (1×) (Gibco) for approximately 4 minutes at 37° C. Single cell suspensions were spun down, resuspended in DMEM-high glucose/10% FCS/1% PenStrep and replated in new culture flasks (Corning). Cells were kept at 37° C. in a 5% CO₂ atmosphere.

iPSC Cultures

The iPSC line used is referenced as LUMC0044iCtrl44 (Chen et al. Nat Commun. 2017). The iPSC line LUMC0044iCtrl44 was genetically modified to contain a construct of DOX-inducible coFOXN1 and a T2A-P2A-mScarlet reporter inserted in the AAVS1 safe harbor locus. Several genetically modified clones were obtained, of which two were used and further characterized. These two clones are designated coFOXN1mSc-2 and coFOXN1mSc-4 (or, together, coFOXN1mSc) hereafter.

iPSC LUMC0044iCtrl44, coFOXN1mSc-2 and coFOXN1mSc-4 were cultured in 6-well plates (Corning) coated with Geltrex™ LDEV-Free (Gibco (ThermoFischer)) in mTeSR Plus (STEMCELL Technologies) according to the manufacturer's instructions. Cells were passaged as clumps using Gentle Cell Dissociation Reagent (STEMCELL Technologies) every 5 to 7 days, when uniform and tightly packed colonies with a multilayered core and single-layered edges were visible. Cells were cultured in a 37° C. 5% CO₂ incubator.

Differentiation of iPSCs

Definitive endoderm (DE) was induced using the STEMdiff™ Definitive Endoderm Kit (STEMCELL Technologies). In brief, iPSCs were seeded at a density of 0.18 million cells/cm² in mTeSR plus+10 μM Y-27632 (Dihydrochloride) ROCK Inhibitor (RI) (STEMCELL Technologies) (day 0) and subsequently differentiated using the DE kit culture media (days 1 to 5). Modifications to the protocol provided by the kit are stated in the results section. For instance, different incubation times were tested for the DE induction media. Replating of DE cells was achieved using 150 000 cells per 48 well plate. For 1 day after replating, RI was supplemented to the medium at 10 μM.

After differentiation to DE, cells were cultured in a base medium of DMEM/F-12 with 15 mM HEPES (STEMCELL Technologies)+B-27™ Supplement, serum free (Gibco (ThermoFisher)) (1:200 ratio). For the induction of AFE, either LY364947 (LY) (5 μM)+rhBMP4 (50 ng/mL)+EC23 (RA) (0.25 μM) (protocol A) or LY (5 μM)+Dorsomorphin (2 μM) (protocol B) was added for 3 or 4 days. Protocol B was used for all iTEPC differentiations after optimization of AFE differentiation.

The standard protocol for induction of PPE (PPE A) consisted of LY (5 μM)+rhFGF8 (50 ng/mL)+EC23 (0.1 μM)+SAG (Smoothened Agonist) (100 ng/mL) added to the base medium for 2 days after induction of AFE. Several additional media for PPE induction were tested, with either a 2-day or 4-day PPE induction period, as listed in Table 1. Molecules in the table were used at the following concentrations, unless indicated otherwise: LY (5 M), rhFGF8 (50 ng/mL), EC23 (0.1 μM), SAG (100 ng/mL), Dorsomorphin (1 μM), Cyclopamine-KAAD (0.5 μM), rhWNT3a (50 ng/mL), rhBMP4 (50 ng/mL). After PPE induction, cells were incubated in a TEPC induction medium composed of Cyclopamine-KAAD (0.5 μM)+rhWNT3a (50 ng/mL)+rhBMP4 (50 ng/ml) for 5 days to direct the cells to an iTEPC fate. Noggin (6057-NG/CF) was used at 200 μg/mL and SB431542 at 10 μM as alternative for the AFE medium.

TABLE 1
Pharyngeal pouch endoderm (PPE) induction
media tested (2-day and 4-day protocols)

| | PPE | PPE | PPE | PPE |
|---|---|---|---|---|
| | induction | induction | induction | induction |
| | medium | medium | medium | medium |
| Protocol | day 1 | day 2 | day 3 | day 4 |
| Protocol A | LY + rhFGF8 + EC23 + SAG | | X | X |
| (standard) | | | | |
| Protocol B | LY + rhFGF8 + EC23 + | | X | X |
| | SAG + Dorsomorphin | | | |
| Protocol C | LY + rhFGF8 + | LY + rhFGF8 + | X | X |
| | EC23 + SAG | EC23 | | |
| | (400 ng/mL) | | | |
| Protocol D | Cyclopamine-KAAD + | | X | X |
| | rhWNT3a + rhBMP4 | | | |
| Protocol E | LY + rhFGF8 + EC23 + SAG | | | |
| Protocol F | LY + rhFGF8 + EC23 + | | LY + rhFGF8 + | |
| | SAG + Dorsomorphin | | EC23 + SAG | |
| Protocol G | LY + FGF8 + | LY + rhFGF8 + EC23 | | |
| | EC23 + SAG | | | |
| | (400 ng/mL) | | | |

| | |
|---|---|
| Protocol H | Cyclopamine-KAAD + rhWNT3a + rhBMP4 |

DOX Treatment of iPSCs

iPSCs were treated with 1 μM DOX (Hyclate) (STEMCELL Technologies) for 24 hours. DOX treatment (1 μM) during differentiation was included in the 5-day TEPC induction medium when stated.

### RT-qPCR

Cells for RT-qPCR were preserved in RLT buffer (provided by the RNeasy microkit (Qiagen)) with 1% β-mercaptoethanol (Sigma), following the RNeasy kit manufacturer's instructions. Samples were stored at −80° C. until further analysis. Briefly, RNA was isolated using the RNeasy microkit (Qiagen). cDNA synthesis was done using SuperScript III Reverse Transcriptase (Invitrogen), Oligo (dT) 15 and random primers. RT-qPCR was carried out with Taqman fast advanced mmix (Life technologies) on the Quantstudio 3 Real-Time PCR System (Thermofisher Scientific). ACTINB was used as a housekeeping gene for all experiments. For the primers used, see Table 2.

TABLE 2
RT-qPCR primer information

| Target | Company | Assay id or primer sequence |
|---|---|---|
| ACTINB | Thermofisher | Hs01060665_g1 |
| coFOXN1 | X | FW: 5′TATGACCGAGCACTTCCCCT- |
| | | 3′ |
| | | RV: 5′-TCAGAGACAGGTTATGCCG |
| | | C-3′ |
| DLL4 | Thermofisher | Hs_00184092_m1 |
| EYA1 | Thermofisher | Hs00166804_m1 |
| FOXN1 | Thermofisher | Hs00919266_m1 |
| HOXA3 | Thermofisher | Hs00601076_m1 |
| PAX1 | Thermofisher | Hs01071293_g1 |
| PAX9 | Thermofisher | Hs_00196354_m1 |
| TBX1 | Thermofisher | Hs00962558_g1 |

### Spectral Flow Cytometry

Spectral Flow Cytometry of iTEPCs and Artificial Thymic Organoids (ATOs)

For flow cytometric analysis, cells were harvested either with 0.05% Trypsin-EDTA (MS5-hDLL4) or TrypLE™ Express Enzyme (1×), no phenol red (Gibco) (iPSC-derived cells). ATOs were harvested by pipetting the ATO in a drop of PBS using a 1 mL pipet tip. ATOs were pipetted onto a Falcon® Snap Cap (Corning) and hematopoietic cells were forced through the filter by washing with PBS and gentle scraping with a 1 mL pipet tip. ATOs in Geltrex were first incubated in Collagenase/DNase-I to digest the matrix before putting the cells through the filter. Collagenase/DNase-I consisted of 2 mg/mL Collagenase (Roche) and 0.1 mg/mL DNase-I (Merck) in RPMI 1640 (Gibco). For cell surface staining, cells were washed with FACS buffer (PBS/0.1% Azide/0.2% BSA), incubated in antibody mix for 30 min at 4° C., washed with FACS buffer, and kept on ice or 4° C. until final analysis.

All samples were analyzed on the Cytek® Aurora 5L spectral flow cytometer. For each measurement, unstained cells of the same type as the measured sample were included as a control. For single stained reference controls we used single stained beads (UltraComp Beads, BD Science). As a reference control for mScarlet, iPSCs with DOX were used, which showed a brighter signal than the differentiated cells with DOX. Antibodies used are listed in Table 3.

### Human Thymus Material

Staining of human TECs was done on isolated thymic stromal cells using the protocol from Stoeckle et al. (J Vis Exp. 2013) until the second digestion step. Human thymus material was obtained from children undergoing cardiac surgery, with written informed consent from the parents.

TABLE 3
Antibodies used for flow cytometry

| Antibody | Conjugate | Clone | Company | Article nr | Dilution |
|---|---|---|---|---|---|
| CD7 | 124-1D1 | Alexa Fluor 532 | Thermo Fisher Scientific | 58-0079-42 | 1:100 |
| CD19 | SJ25C1 | Alexa Fluor 647 | Biolegend | 363040 | 1:400 |
| CD33 | Sn428 | Test vial | Beckman Coulter | Test vial | 1:100 |
| CD45 | BV650 | HI30 | BD | 563717 | 1:200 |
| CD56 | APC-Cy7 | HCD56 | Biolegend | 318332 | 1:400 |
| CD117 | PE-Cy7 | 104D2D1 | Beckman Coulter | B49221 | 1:200 |
| CD184 | PE-Cy5 | 12G5 | Biolegend | 306508 | 1:1600 |
| CD205 | PerCP-Cy5.5 | HD30 | Biolegend | 342210 | 1:200 |
| CD271 | APC | ME20.4 | Biolegend | 345107 | 1:100 |
| DLL4 | APC | MHD4-46 | Biolegend | 346507 | 1:400 |
| EPCAM | APC-Cy7 | 9C4 | Biolegend | 324245 | 1:400 |
| PDPN | PE-Dazzle594 | NC-08 | Biolegend | 337027 | 1:800 |

### 3D Cultures: Spheroids

Spheroids were formed in 96-well round-bottom ultra-low attachment plates with AFE stage cells. A single-cell suspension was made; the cells were then spun and resuspended in PPE medium+RI. 16,500 cells were seeded into each well with 100 uL of PPE medium and spun down at 300 g for 1 minute. On the next day, a media change was performed to remove RI. Individual spheroids at the PPE stage were grouped together in sets of 10-20 spheroids within the 96-well plate. The medium was carefully removed. Each cluster of spheroids was then transferred to a 2 mL Eppendorf tube, where they were mixed and resuspended with 20 μL of Geltrex. Next, a single droplet of the mixture was placed onto the insert membrane of the air-liquid interface within a 6 or 12-well plate. The plate was inverted and incubated at a temperature of 37° C. for a duration of 20-30 minutes. Underneath each insert, TEC medium was added.

### Artificial Thymic Organoid (ATO) Formation

### MS5-ATOs

ATO formation was done following the published protocol (Seet et al. Nat Methods. 2017). In brief, human CD34+ cells were thawed on the day of ATO formation and counted using a hemocytometer. MS5-hDLL1 cells were harvested and counted. CD34⁺ cells and MS5-hDLL1 were mixed at a ratio of 1:20 (7.500 CD34⁺ cells to 150.000 MS5-hDLL1 cells), spun down, resuspended either in ATO medium or Geltrex as indicated, and placed onto a 0.4 μm Millicell Cell Culture Insert (Merck/Millipore; PICM0RG50). ATO base medium deviated slightly from the published protocol and was composed of DMEM/F-12+B-27TM (1:200)+30 μM ascorbic acid (Sigma)+1% PenStrep and was stored for a maximum of 2 weeks at 4° C. On the day of medium change, 5 ng/ml hIL7 and 5 ng/mL hFLT3 were added. Medium was changed twice per week.

iTEPC ATOs

iTEPC ATOs were formed similarly to the MS5-ATOs described above, where the MS5hDLL1 cells were replaced by the differentiated iTEPCs. On the first day of differentiation, 10 μM Y-27632 RI was added to the medium to promote survival of the iTEPCs.

Optimized iATO-Like Culture

The process for forming iATOs was very similar to the MS5-iATO described above. Briefly, first, human CD34⁺ cells are thawed on the day of ATO formation and counted. iTEPCs were harvested with TrypLE and counted. Then CD34⁺ and iTEPCs were mixed at a ratio of approximately 1:3 (consisting of 60 k CD34⁺ cells to 200 k iTEPCs). The resulting mixture was spun down and resuspended in Geltrex to create drops of 1.5 μL containing 260 k cells. These drops were placed onto an air-liquid cell culture insert into a 6-well or 12-well plate.

The optimized iATO base medium was composed of DMEM/F-12+B-27™ (1:25)+ascorbic acid (30 μM) (Sigma, Cat #A8960-5G)+PenStrep (1%)+human IL7 (50 ng/ml) (Miltenyi Biotec, Cat #130-093-937)+human Flit3 (50 ng/mL) (Miltenyi Biotec)+Stem cell factor (SCF) (50 ng/mL) (Miltenyi Biotec, Cat #130-096-692). This medium was freshly prepared for media changes twice a week. On the first day after formation, 10 μM Y-27632 RI was added to the iATO medium to promote the survival of the iTEPCs. The flow panel below in Table 4 was used to assess the immune population in the iATO.

TABLE 4
flow panel to assess immune population in iATOs

| Antibody | Conjugate | Clone | Company | Cat# | Dilution |
|---|---|---|---|---|---|
| CD5 | V450 | UCHT2 | BD Biosciences | 561154 | 1:200 |
| CD7 | Alexa Fluor 532 | 124-1D1 | Thermo Fisher Scientific | 58-0079-42 | 1:100 |
| CD19 | Alexa Fluor 647 | SJ25C1 | Biolegend | 363040 | 1:400 |
| CD33 | Sn428 | Test vial | Beckman Coulter | Test vial | 1:100 |
| CD33 | BV785 | WM53 | Biolegend | 303428 | 1:200 |
| CD34 | BV510 | 581 | Biolegend | 343527 | 1:25 |
| CD45 | BV650 | HI30 | BD Biosciences | 563717 | 1:50 |
| CD56 | APC-Cy7 | HCD56 | Biolegend | 318332 | 1:400 |
| CD117 | PE-Cy7 | 104D2D1 | Beckman Coulter | B49221 | 1:200 |
| CD184 | PE-Cy5 | 12G5 | Biolegend | 306508 | 1:1600 |
| CD205 | PerCP-Cy5.5 | HD30 | Biolegend | 342210 | 1:200 |
| CD271 | APC | ME20.4 | Biolegend | 345107 | 1:100 |
| DLL4 | APC | MHD4-46 | Biolegend | 346507 | 1:400 |
| EpCAM | APC-Cy7 | 9C4 | Biolegend | 324245 | 1:400 |
| EpCAM | BV421 | 9C4 | BioLegend | 324219 | 1:100 |
| HLA-DR | Alexa Fluor 700 | G46-6 | BD Bioscience | 560743 | 1:100 |
| OCT3/4 | BV421 | 40/Oct-3 | BD Biosciences | 565644 | 1:25 |
| Podoplanin | PE-Dazzle594 | NC-08 | Biolegend | 337027 | 1:800 |
| Podoplanin | APC/Cy7 | NC-08 | Biolegend | 337029 | 1:400 |
| SOX2 | Alexa Fluor 647 | 14A6A34 | Biolegend | 656108 | 1:25 |

### Statistics

qPCR data were analyzed using one-way ANOVA, with Turkey's multiple comparisons, or two-tailed student's T test, as specified in the figure legends.

### Mice

4- to 5-week-old Balb/C-nude (BALB/cAnN-Foxnlnu/nu/Rj) mice were obtained from Janvier Laboratories (Le Genest Saint Isle, France). NSG-nude (NOD.Cg-Foxn1em1DvsPrkdcscidIl2rgtm1Wjl/J) mice were purchased from the Jackson Laboratory (Bar Harbor, Maine, USA) and bred at the animal facility of the Leiden University Medical Center. All mice were maintained under sterile and specific pathogen-free conditions. All mouse experiments were performed in accordance with institutional and national guidelines of the Central Committee Animal Experiments (Centrale Commissie Dierproeven) and were approved by the Animal Welfare Body of the Leiden University Medical Center.

iThymus Organoids

iTEPCs and MSCs were harvested with TrypLE and counted. Then MSCs and iTEPCs were mixed at a ratio of 50 k MSC cells to 200 k iTEPCs. The resulting mixture was spun down and resuspended in drops of 1.5 μL containing 250 k cells. These drops were placed onto an air-liquid cell culture insert into a 6-well or 12-well plate overnight with DMEM/F12+B27 (1:200) and rock inhibitor (RI). For the NSG-nude model, 15 k purified primed CD34+ were added per organoid. Priming of CD34+ was performed for 3 days according to protocol described by Montel-Hagen, Amélie, et al. (Cell reports 33.4 (2020)).

### Kidney Capsule Transplantation

Recipient mice were anesthetized with isofluorane and received a subcutaneous (s.c.) injection (0.3 mg/ml) of buprenorphine (Temgesic, Schering-Plough, Belgium) for pain relief. The right kidney was exteriorized via flank incisions and a small incision was made in the renal capsule. iThymus organoids (n=6 per kidney for Balb/C-nude and n=9 per kidney for NSG-nude) were transplanted under the kidney capsule by using a siliconized polyethylene tube. After transplantation, the peritoneum and skin were sutured. Next, mice were immediately treated with buprenorphine s.c. and placed in a cage on a heating pad to recover.

### Preparation of Mouse Tissue and Flow Cytometry

To monitor T cell development in live Balb/C-nude mice over time, peripheral blood was collected at specified intervals via tail vein puncture in Microvette CB300 LH tubes (Sarstedt). Red blood cells were lysed by incubation with NH4C1 8.4 g/l; KHCO3 1 g/l, (pH 7.4) twice for 1 min at room temperature. The lysis activity was neutralized by adding FACS buffer (pH 7.4/0.2% BSA/0.1% NaN3) and cells were stained as described below. For flow cytometry, surface marker staining was performed by incubation with an antibody mix for 30 min in FACS buffer. Subsequently, cells were fixed by using the FOXP3 Transcription Factor Staining Buffer Set according to the manufacturer's protocol (Thermo Fischer Scientific). Dead cells were excluded by using Fixable Viability Near-infra Red Dye (1:1000, Life Technologies). Fluorescence minus one (FMO) was used as a negative control for T cell development markers. Single stained controls of immunocompetent Balb/C splenocytes or single-stained beads (UltraComp Beads, BD Biosciences) were used for reference controls. Samples were analyzed on the Cytek® Aurora 3L spectral flow cytometer. All generated data were analyzed using the OMIQ software (Dotmatics, Boston, MA). The antibodies used are listed below in Table 5.

TABLE 5
Antibodies for flow cytometry

| Marker | Fluorchrome | Clone | Company | Cat. No. | Dilution |
|---|---|---|---|---|---|
| CD19 | BV421 | 6D5 | BioLegend | 115537 | 1:400 |
| Ly6C | PB | HK1.4 | BioLegend | 128013 | 1:1000 |
| CD45 | BV510 | 30-F11 | BD Biosciences | 563891 | 1:800 |
| NKp46 | BV605 | 29A1.4 | BD Biosciences | 564069 | 1:200 |
| CD4 | BV711 | GK1.5 | BD Biosciences | 563050 | 1:200 |
| CD44 | BV785 | IM7 | BioLegend | 103041 | 1:100 |
| TCRgd | FITC | GL3 | ThermoFisher | 1-5711-81 | 1:100 |
| CD11b | PE | M1/70 | ThermoFisher | 12-0112-81 | 1:400 |
| CD62L | PE eFluor610 | MEL-14 | ThermoFisher | 61-0621-82 | 1:400 |
| TCRb | PE Cy5 | H57-597 | BD Biosciences | 553173 | 1:800 |
| CD3 | PerCP eFluor710 | 17A2 | ThermoFisher | 46-0032-80 | 1:400 |
| CD8 | PE Cy7 | 53-6.7 | ThermoFisher | 25-0081-81 | 1:800 |
| CD25 | APC | PC61 | ThermoFisher | 17-0251-81 | 1:100 |
| Ly6G | AF700 | 1A8 | BioLegend | 127621 | 1:400 |

### Immunohistochemistry of the Kidney Capsule

Collected mouse kidney tissues were fixed overnight in 4% Paraformaldehyde (Thermo Fisher Scientific, CAT #J19943.K2) before being embedded in paraffin to create formalin-fixed, paraffin-embedded (FFPE) blocks. To be able to perform hematoxylin and eosin histology (H&E) and immunohistochemistry (IHC), FFPE blocks were cut into 4 μM sections using a rotary microtome (Leica Biosystems). Sections were deparaffinized and dehydrated, and endogenous peroxidase activity was blocked. For H&E, sections were stained with hematoxylin (Sigma-Aldrich, CAT #109249) for 5 minutes followed by Eosin Y (Sigma-Aldrich E6003-100G) for 1 minute. For IHC, antigen retrieval was performed in a steamer cooker for 20 minutes at pH6.0 using Citrate Buffer (Thermo Fisher Scientific, CAT #005000). Tissues were stained in 1:150 diluted CD3 antibody (Abcam, CAT #AB16669). Detection was achieved using diaminobenzidine (DAB) chromogen (Agilent, CAT #K346811-2) and counterstaining with hematoxylin. Imaging was done using EVOS™ M7000 Imaging System (Thermo Fisher Scientific, CAT #AMF7000).

### Example 1

Characterization of a DOX-Inducible FOXN1 iPSC Cell Line

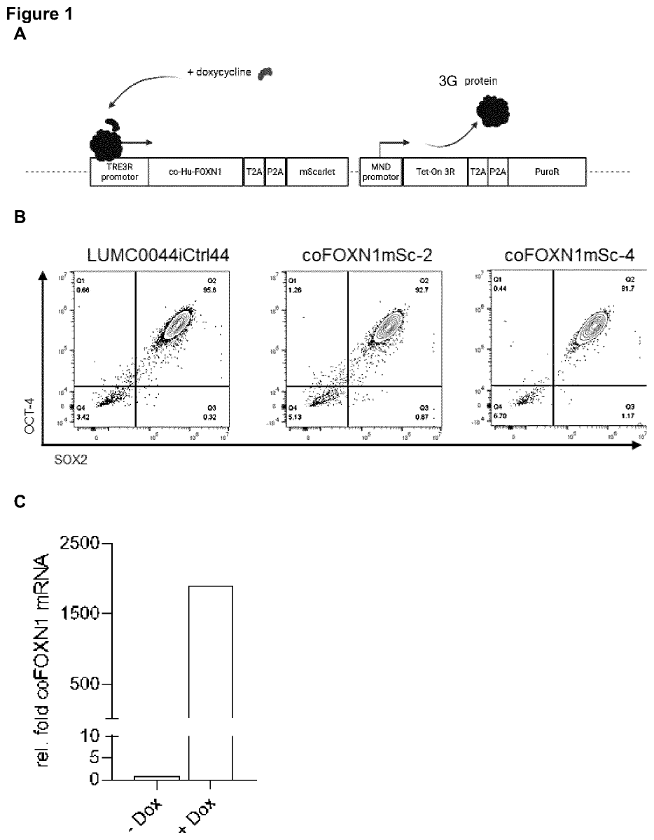

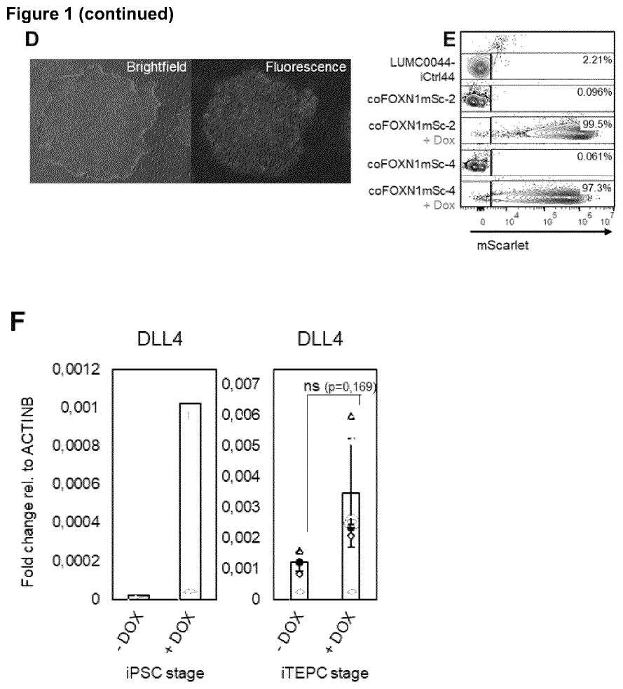

We used the induced pluripotent stem cell (iPSC) line LUMC0044iCtrl44 and two genetically modified clones derived from this cell line that contain a doxycycline (DOX)-inducible construct of codon optimized FOXN1 (coFOXN1) and an mScarlet reporter (FIG. 1A). In the codon optimized FOXN1 (coFOXN1), at least one CpG island and at least one cryptic splice site is removed and codon optimized for an efficient fit with human tRNAS, aiming to enhance the protein expression processes.

Here, we show that, without DOX, coFOXNmSc-2 and coFOXNmSc-4 expressed pluripotency markers OCT4 and SOX2 at levels similar to the LUMC0044iCtrl44 line (FIG. 1B), demonstrating no loss of pluripotency. With DOX, coFOXN1mSc iPSCs expressed coFOXN1 mRNA (FIG. 1C) and produced mScarlet protein (FIG. 1D). Quantification of mScarlet expression by flow cytometry (FC) showed that almost 100% of the iPSCs were mScarlet⁺ when treated with DOX for 24 hours (FIG. 1E). Without DOX, coFOXN1mSc iPSCs did not show mScarlet expression, similarly to the parental LUMC0044iCtrl44 line. This suggests that there is almost no leakiness of the system and that the coFOXN1mSc iPSCs without DOX can serve as a control for those with DOX. To assess the functional effects of turning on coFOXN1 expression, we monitored mRNA expression levels of the FOXN1 target DLL4 (Žuklys et al. Nat Immunol. 2016). The induction of coFOXN1 at the iPSC stage led to an increase in DLL4 mRNA (FIG. 1F). The upregulation of DLL4 mRNA with DOX suggests that, with DOX, functional FOXN1 protein likely is present. This indirect indication of FOXN1 protein is also important since no reliable antibody for the detection of FOXN1 is currently available. Together, these data show that coFOXN1mSc iPSCs retain pluripotency markers and can be induced to express the desired construct (by the presence of mScarlet protein and coFOXN1 mRNA). Thus, these data show that we successfully designed and developed an inducible system for coFOXN1 expression.

Demonstration of the Proper Integration within the Safe Harbor Locus AAVS1.

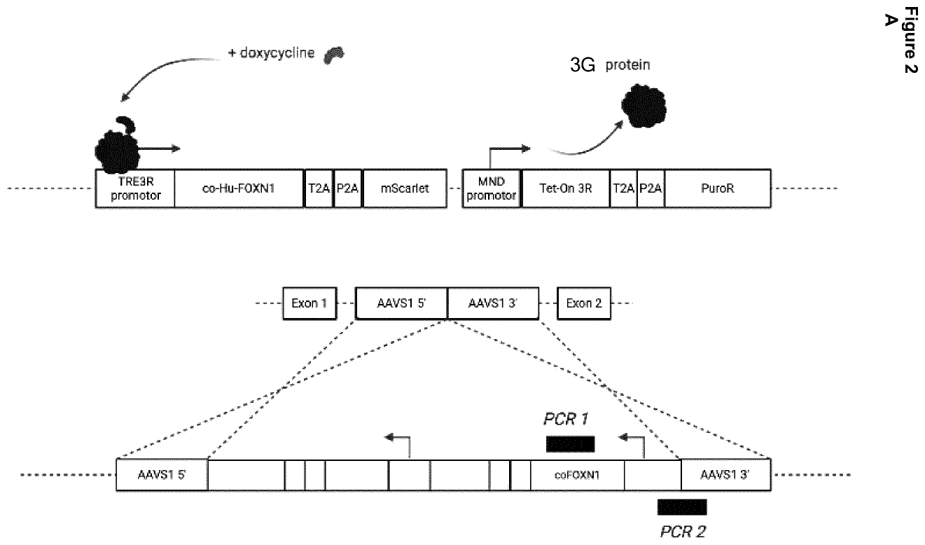

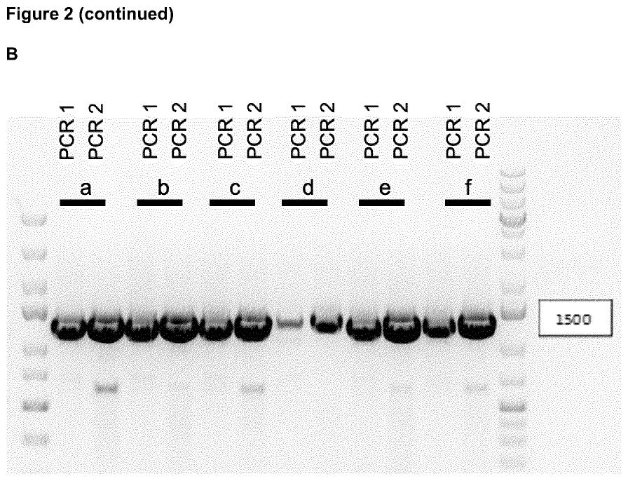

To ensure that the coFOXN1mSc clones do not only contain the construct, but have it correctly integrated in the right locus, we performed genomic PCR (FIG. 2). The genomic PCR strategy is designed to ensure that the coFOXN1 is present in the genomic DNA (see PCR 1 primers) and that the safe harbor locus AAVS1 is connected to the construct (PCR 2 primers). The PCR products generated by this technique both have an expected size of 1500 bp. All coFOXN1mSc clones (a to f) have fragments for both PCR of 1500 bp, demonstrating the proper integration within the right DNA locus (FIG. 2B).

Inhibition of TGFβR1 and BMP Following DE Differentiation Induced a High Percentage of CD56⁺CD271⁺ DP AFE Cells

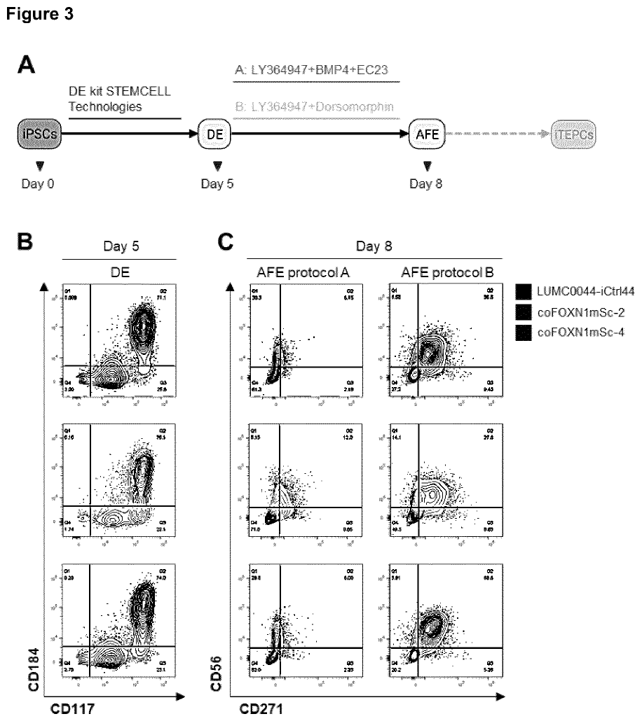

Before employing the DOX-inducible system, we set out to optimize a directed differentiation protocol that could be used in combination with coFOXN1 induction. Based on previously published—but contradicting—protocols to induce anterior foregut endoderm (AFE) (Parent et al. Cell Stem Cell. 2013; Zeleniak et al. Nat Methods. 2022; Gras-Peña et al. J Allergy Clin Immunol. 2022; Green et al. Nat Biotechnol. 2011), we performed a side-by-side comparison of two protocols (A and B; FIG. 2A) to direct iPSCs to AFE. iPSCs were first differentiated to definitive endoderm (DE) using the STEMdiff™ Definitive Endoderm kit for 5 days. At day 5, FC analysis for DE markers CD117 and CD184 showed similar percentages of double positive (DP) cells for the LUMC0044iCtrl44 line (71.1%), coFOXN1mSc-2 (76.1%) and coFOXN1mSc-4 (74.0%) (FIG. 3B). This indicates that the genetic modification of the iPSC line with the construct does not negatively affect differentiation to DE.

The DE cells were then exposed to either a mixture of the transforming growth factor beta (TGFB) inhibitor LY364947 (LY), BMP4 and RA (retinoic acid) (protocol A) or a mixture of LY and Dorsomorphin (“Dorso”) (BMP inhibitor) (protocol B) for 3 days (FIG. 3A). Using the AFE surface markers CD56 and CD271 as a readout (Brafman et al. Stem cell reports. 2013; Leibel et al. iScience. 2022), protocol B yielded a higher efficiency of CD56+CD271+ DP cells than protocol A for all three iPSC lines tested (FIG. 3C). Therefore, we decided to use protocol B (dual inhibition of TGFB and BMP) for all subsequent differentiation experiments.

### Further Characterization of BMP Activation/Inhibition: CD56+ CD271+ Marks SOX2+ Cells

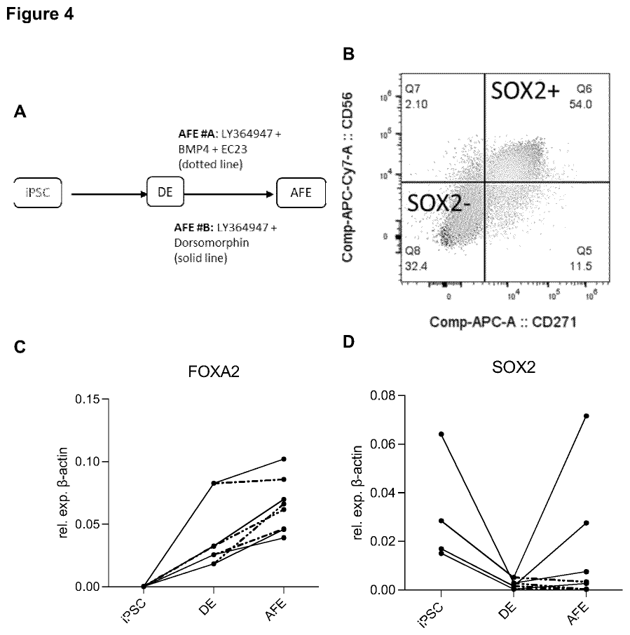

We optimized an intracellular staining for SOX2 on flow cytometry and showed that CD56+ CD271+ double positive cells are also SOX2+ (FIG. 4). SOX2 is a widely used marker to describe AFE stage and is usually used in combination with FOXA2. To strengthen our data comparing protocol A and B, we performed gene expression analysis at iPSC, DE and AFE stages. FOXA2 was increased in both protocol A and B, showing that the cells are correctly directed toward a definitive endoderm stage. Looking at SOX2, we clearly see a re-upregulation of SOX2 from DE to AFE stage only for the protocol B. These data suggest that protocol B reached higher AFE efficiencies based on key transcriptional factors as well as surface markers (FIG. 3).

### Optimization of the Timing of AFE Induction

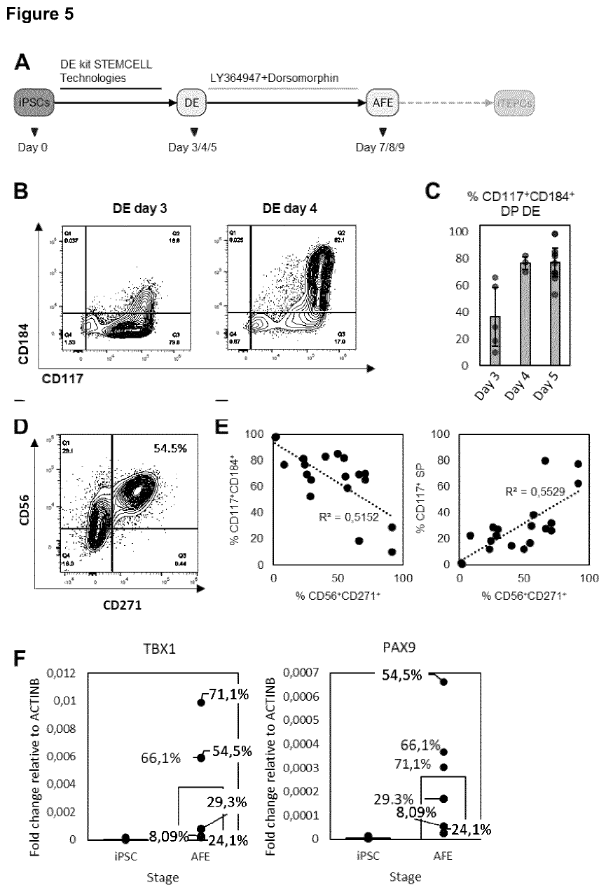

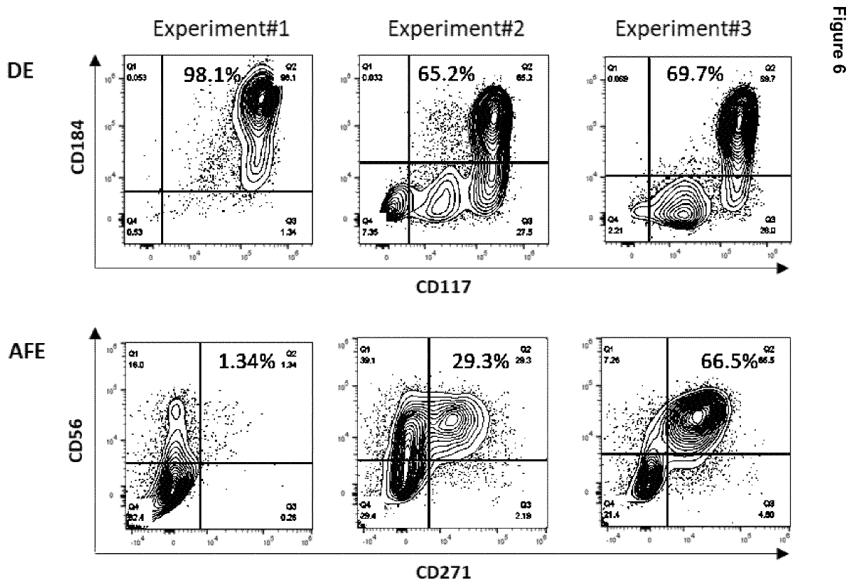

We optimized timing of AFE induction since we observed a high variability in the efficiency across experiments. To test this, cells were differentiated to DE for either 3, 4 or 5 days before being exposed to the AFE induction medium (FIG. 5A). Cells progressing to DE first became CD117⁺ SP, and then CD117 CD184⁺ DP, as shown by DE marker expression on two subsequent days within one experiment (FIG. 5B) and quantified in FIG. 5C. We showed next that using the AFE markers (FIG. 5D), all independent differentiations lead to AFE cells with varying efficiencies (FIGS. 5E and 6). However, the protocol requires some further optimization as a negative trend is observed between high percentage of CD117⁺CD184⁺ DP cells at the DE stage and high percentage of CD56⁺CD271⁺ DP. Importantly, experiments with a high percentage of CD56⁺CD271⁺ DP cells tended to cluster at higher expression of AFE transcriptions factor PAX9 and TBX1 than experiments with a low percentage of CD56⁺CD271⁺ DP cells (FIG. 5F). Percentage depicted in the FIG. 5F indicates the AFE percentage (CD56+ CD271+ DP) of the specific differentiation. Together, these data suggest that stimulation of DE cells SP for CD117 can efficiently generate an AFE population expressing the markers CD56 and CD271. It may be important to have a SP-CD117+population of at least 30% of the total cell count at the moment of the switch. In the cell mixture there are: SP (single positive) for CD117, DP for CD184 and CD117 or negative cells. So it is preferred to have about: 30% SP CD117+ and 70% of DP CD117+CD184+. Efficiency is affected if there are less than 30% SP CD117 and more than 70% of DP CD117+CD184+.

### Replating DE Cells at Lower Density Overcomes Low AFE Efficiencies

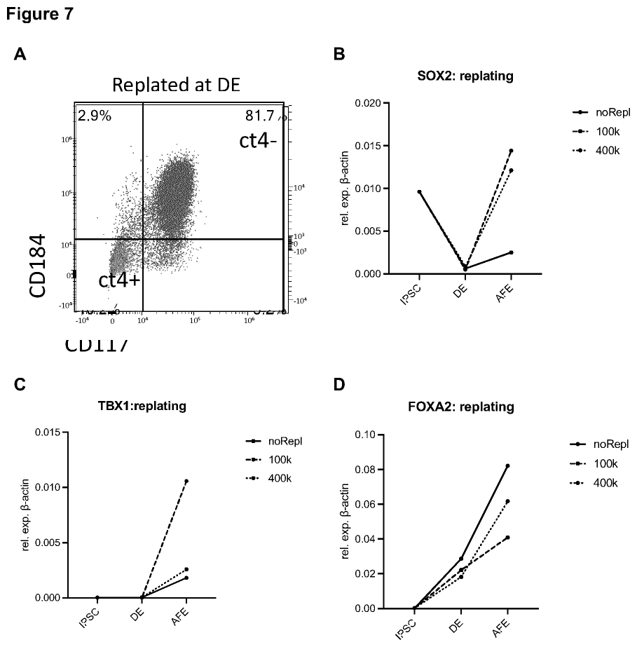

We further improved the DE-AFE transition by replating the DE cells at a lower density 1 day before the DE stage with the addition of Rock inhibitor (FIG. 7). This small optimization allows the DE % to be higher than 80% (purer population) and having high SOX2+ percentage present at AFE stage. Gene expression analysis shows how replating positively affects the expression of SOX2 at the AFE stage, while FOXA2 remains unchanged.

AFE Protocol B Reached Higher Percentages of AFE Cells with SB431542 (SB) and Noggin

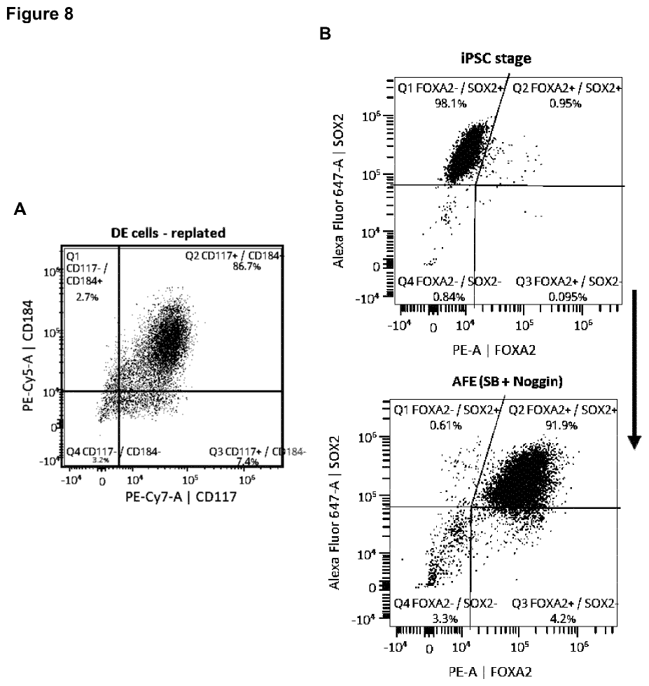

Next, we assessed alternative small molecules and reagents to LY and Dorsomorphin, such as SB431542 (SB) and Noggin. SB431542 is known as a selective inhibitor of the TGF-b type 1 receptor and should have the same effect as LY. Noggin is an inhibitor of several BMPs and is expected to have the same effect as Dorsomorphin. Using the SB+Noggin combination, AFE cells that are FOXA2+ SOX2+ were successfully generated as showed by FC (FIG. 8). Thereby, differentiation to FOXA2+ SOX2+ AFE cells has been optimized.

Differentiated iTEPCs Expressed HOXA3, DLL4 and FOXN1 after Directed Differentiation

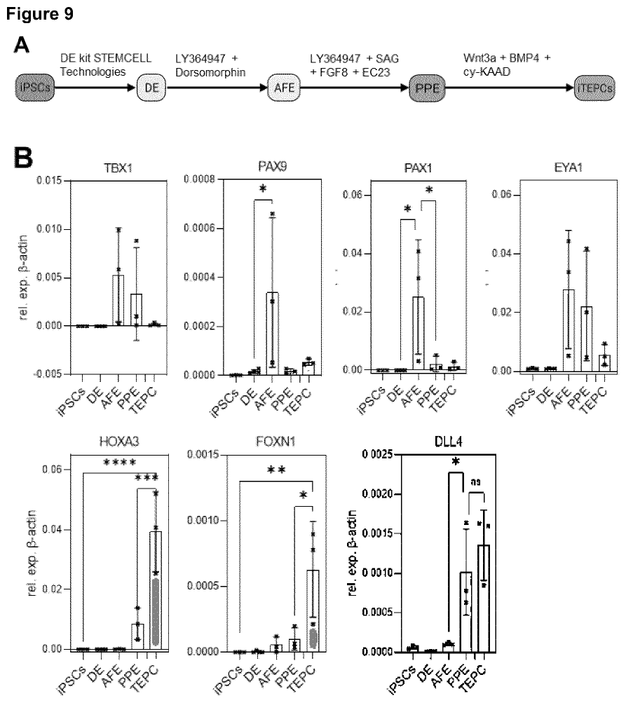

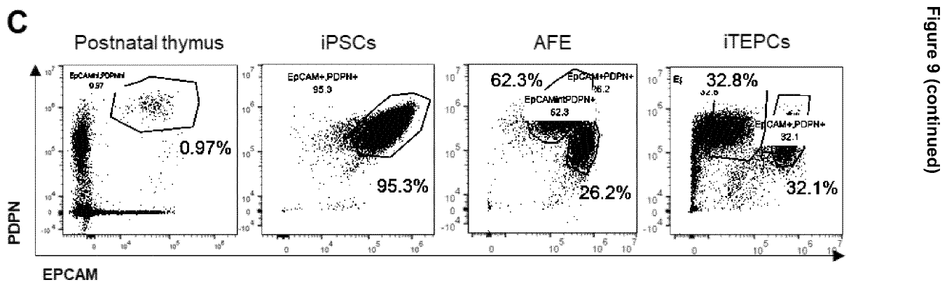

We developed an iTEPC differentiation protocol based on previously published protocols (Chhatta et al., supra) and based on what is known from thymus development in vivo (see FIG. 9A and methods). The directed differentiation protocol induced expression of HOXA3, DLL4 and FOXN1 at the iTEPC stage compared to iPSC stage, with the main increase occurring from the PPE stage to the iTEPC stage (FIG. 9B). Expression of AFE markers TBX1, PAX9, PAX1 and EYA1 peaked at AFE stage (FIG. 9B). A decrease in TBX1 is likely desired after the PPE stage (Reeh et al. Development. 2014).

To assess the phenotype at a single cell level, we performed FC for TEC surface markers. Within the postnatal thymus, TECs can be distinguished from other cell types by FC as being CD45−/lo cells expressing epithelial cell adhesion molecule (EPCAM) and podoplanin (PDPN) (Haunerdinger et al. Front Immunol. 2021), We found approximately 1% of TECs in thymus material enriched for stromal cells (FIG. 9C), which is in line with the low frequency of TECs in total thymus (Stoeckle et al. J Vis Exp. 2013). Of note, EPCAM and PDPN were also present on our iPSCs (FIG. 9C). Interestingly, three populations emerged based on the expression of EPCAM and PDPN during the differentiation to iTEPCs (FIG. 9C).

High Reproducibility of the iTEPC Protocol: Combination of More than 10 Independent Differentiations

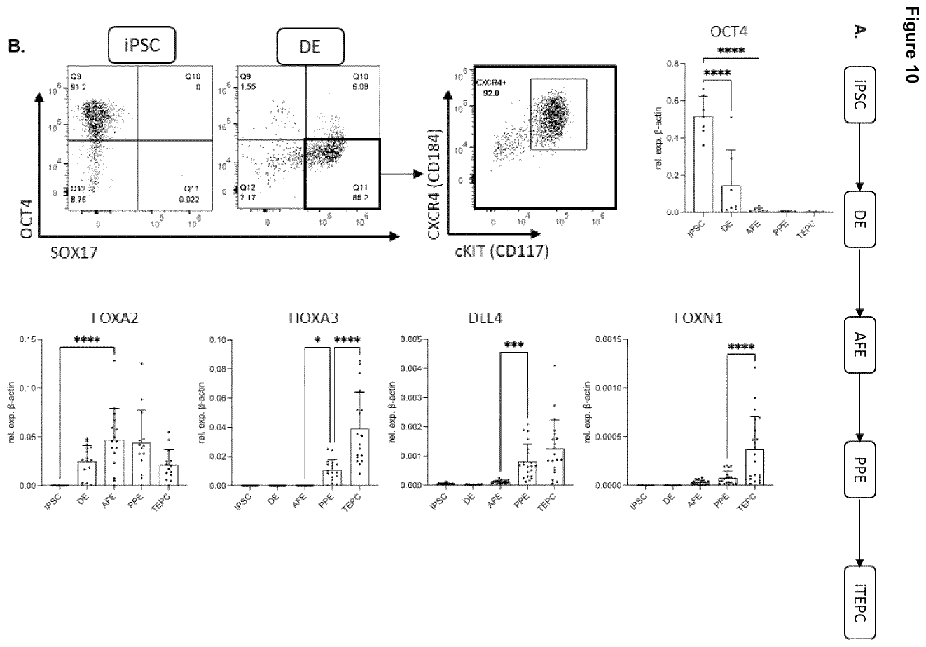

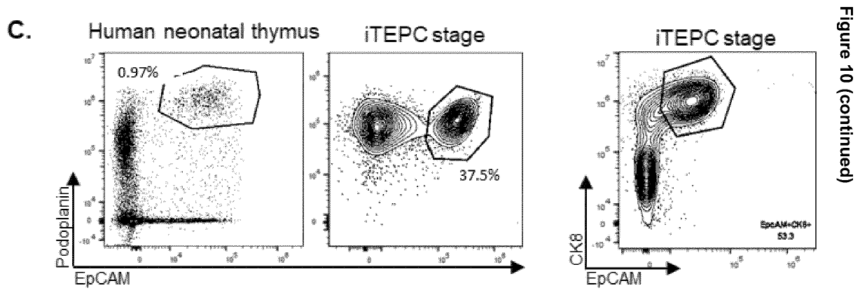

To show reproducibility of the protocol, we combined a large number of experiments' gene profiles together and performed statistical analysis (one-way ANOVA) (FIG. 10). FOXA2 shows the expected upregulation from DE stage and is prolonged throughout the differentiation. Protein and gene expression analysis per stage showed (1) high efficiency in generating DE cells, (2) loss of pluripotency overtime, (3) FOXA2-HOXA3 increase which leads to (4) FOXN1 and DLL4 increase. Clear statistical significance is also shown for HOXA2, DLL4 and FOXN1. The iTEPCs retained a podoplanin+ EpCAM+ population, which is also observed in the postnatal thymus. In addition, we stained for cytokeratin-8 (CTK8), which a key cytokeratin expressed in thymic progenitor cells. We observed that EpCAM+ cells are CTK8+.

HOXA3 Expression at the iTEPC Stage Required Exposure to a Combination of RA, SAG, LY and FGF8

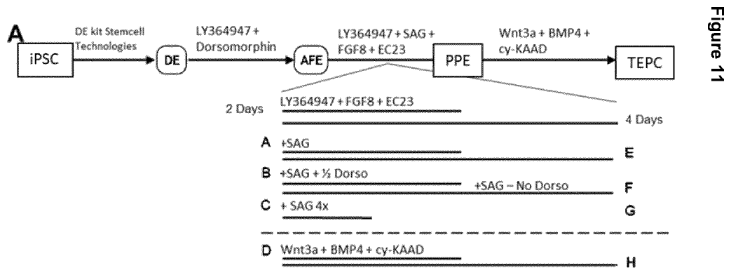

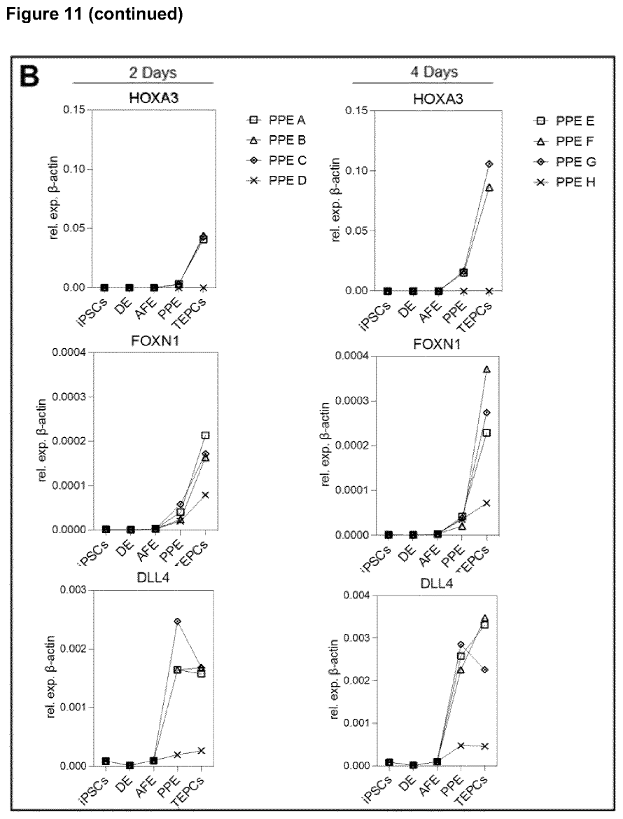

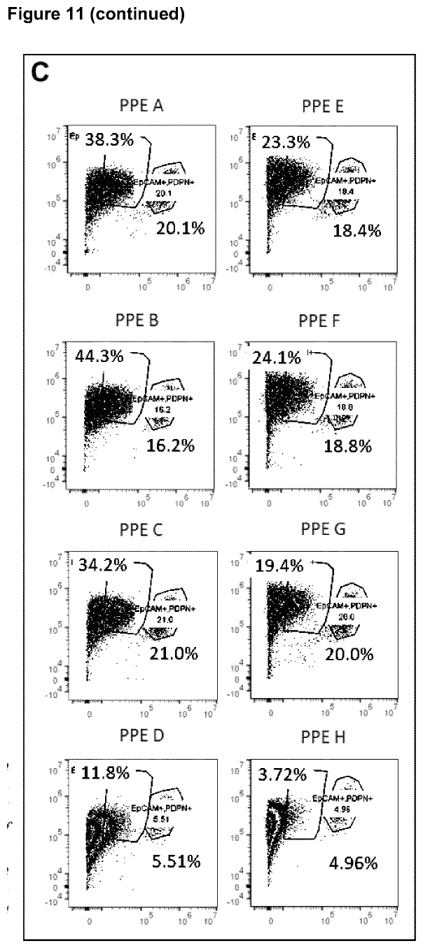

After having established a protocol that induced expression of HOXA3, DLL4 and FOXN1, we tested different PPE induction media (FIG. 11A and methods). We tested a medium in which Dorsomorphin treatment was continued in the PPE induction medium, a medium with a ‘SHH (sonic hedgehog) pulse’ and a protocol in which we switched to the TEPC induction medium right after AFE induction. For all these media, we tested both a 2-day period and a 4-day period before switching to the TEPC induction medium (FIG. 11A, and methods). Cells that were induced with the TEPC medium right after induction of AFE did not upregulate HOXA3 expression, both for the 2-day and 4-day protocol (FIG. 11B). In addition, these cells seemed to have lower FOXN1 and DLL4 expression as compared to the other media tested (FIG. 11B). Interestingly, we also observed lower percentages of EPCAMint and EPCAMhi cells for the two conditions that lacked HOXA3 expression, indicating that these cells indeed differed from the conditions that received a form of the PPE medium (FIG. 11C). Notably, the 4-day protocol seemed to result in higher levels of HOXA3 than the 2-day protocol, although the effects on FOXN1 and DLL4 remain to be determined. D/H media are therefore not optimal, and a clear PPE stage/induction is needed. Media comprising LY, SAG, FGF8 and EC23 (RA) is suitable as PPE medium.

Effects of coFOXN1 Induction During Differentiation on Gene Expression and Surface Markers

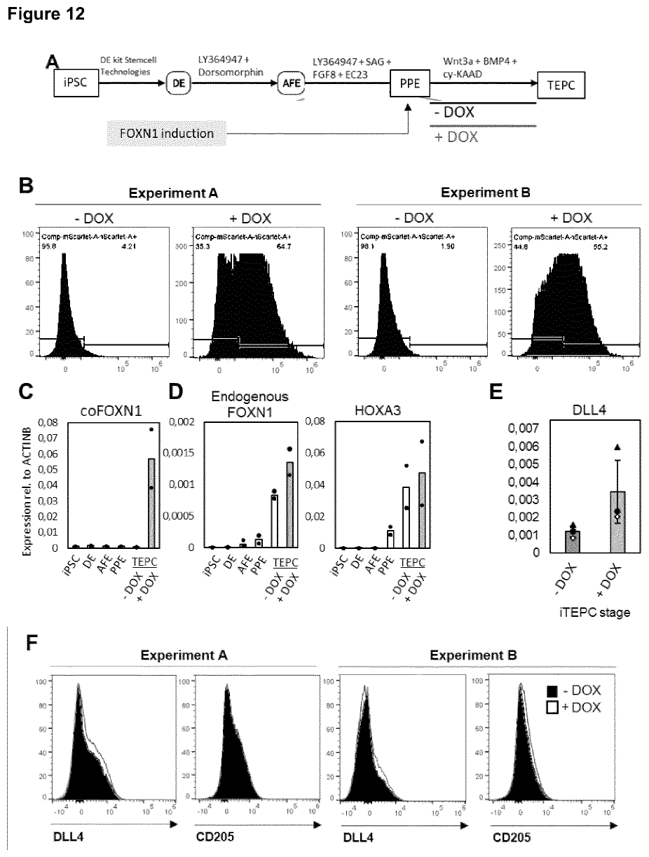

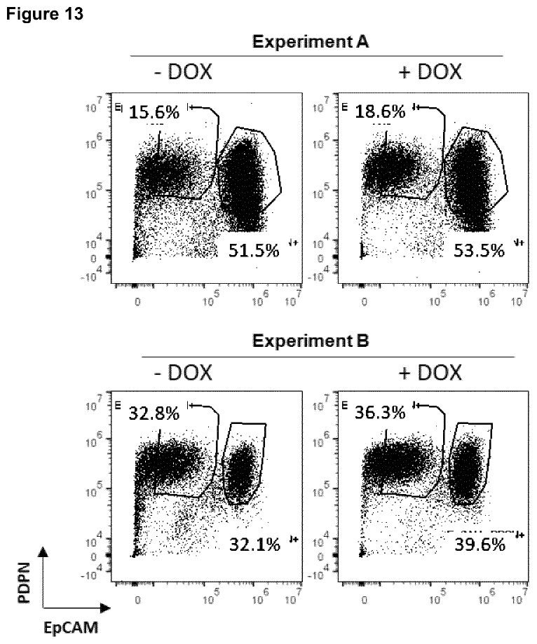

We looked at the effect of coFOXN1 induction starting from the PPE stage onwards, which would most closely mimic the timing during development. Induction resulted in upregulation of coFOXN1 mRNA and mScarlet protein, showing that the AAVS1 safe harbor locus is accessible at the PPE stage (FIG. 12A). Induction with DOX showed presence of the reporter mScarlet by FC (FIG. 12B) and coFOXN1 (FIG. 12C). Over two experiments, DOX did not negatively impact the expression of HOXA3 or endogenous FOXN1 (FIG. 12D). Also by FC no major differences were seen in the levels of PDPN and EPCAM (FIG. 13), nor in the levels of DLL4 and CD205, which is expressed on mature TECs (Jiang et al. Nature. 1995) and during development on bipotent or cTEC progenitors (Shakib et al. J Immunol. 2009; Baik et al. Eur J Immunol. 2013) (FIG. 12F). Importantly, we found DLL4 mRNA to be consistently upregulated in the cells with DOX compared to the cells without DOX (FIG. 12E). This observation also has been shown with FOXN1 expression largely confined to mScarlet+ cells, confirming that DOX induction not only induces mScarlet expression but also FOXN1 and its downstream target gene DLL4.

Technical Replicates Show DOX-Induction Consistently Increased DLL4 and coFOXN1 Expression

Additional independent differentiations were performed (6-12) to test reproducibility (FIG. 14). We observed that mScarlet+ cells are close to 90% at TEPC stage. The TEPCs treated with DOX had significantly higher coFOXN1 and DLL4 on the mRNA level, demonstrating that the coFOXN1 is functional on the protein level.

3D-Culture of iTEPCs Improves DLL4 Expression

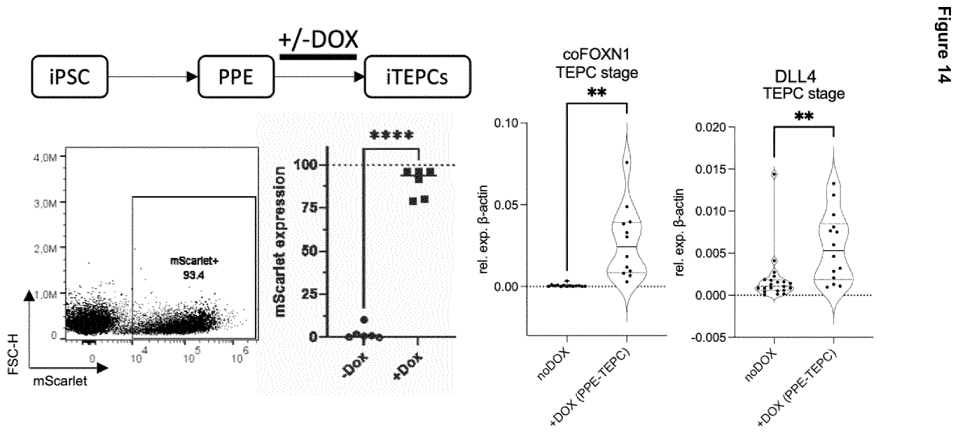

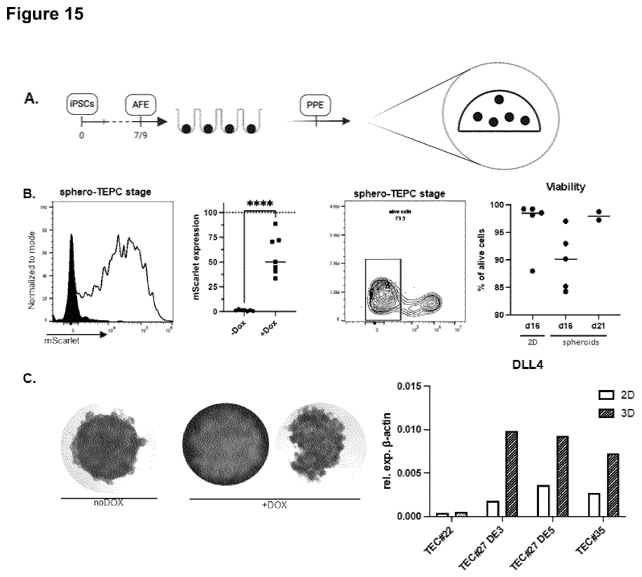

The transition from 2D to 3D is illustrated in FIG. 15A. Briefly, spheroids were generated using AFE cells and transferred on an air-liquid (AL) interface at PPE stage using the commonly used Geltrex™ matrix. In 3D, coFOXN1 (mScarlet+) cells could be induced using DOX, showing that diffusion in the 3D structure does not impair DOX-mediated activation. So-called sphero-TEPCs had high viability at day 21 (FIG. 15B). Sphero-TEPCs showed morphological budding in response to DOX and had higher level of DLL4, highlighting one of the benefits of the 3D culture system (FIG. 15C).

### Standard ATOs Using MS5-hDLL1 Cells Generated in Geltrex™ Supported the Differentiation of HPCs to CD7+CD5+ T-Cell Precursors

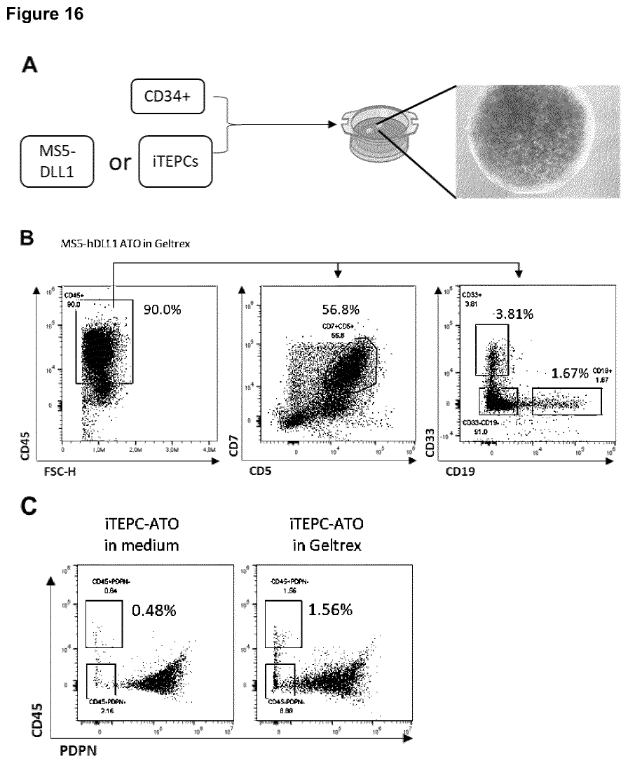

We next assessed the functionality of TEPCs in an ATO co-culture system (FIG. 16A). First, we tested the potential of incorporating an extracellular matrix (ECM) component in the ATO system. Instead of co-culturing the MS5-hDLL1 cells and HPCs resuspended in medium, we resuspended the cells in Geltrex™ basement membrane matrix. HPCs survived within the Geltrex™, as shown by the presence of a CD45+ population within the Geltrex™-ATO after a 3-week coculture (FIG. 16B). The majority of these CD45+ cells showed commitment to the T-cell lineage, by expression of CD7+CD5+. Thus, interaction between the MS5-hDLL1 cells and HPCs is possible within the Geltrex™. A minority of CD33+ cells and CD19+ cells was observed, indicating that a small population started a differentiation program towards the myeloid lineage and B-cell lineage, respectively (FIG. 16B).

Optimization of Co-Culture of iTEPCs and HPCs in an iATO (iTEPC Artificial Thymic Organoid Cultures) System

To test iTEPC functionality in vitro, we performed a first experiment replacing the MS5-hDLL1 cells with our generated iTEPCs without DOX. This pilot study showed that survival of HPCs was higher with Geltrex™ (FIG. 16C).

### Robust HPC Survival is Achieved Using SCF in the ATO System

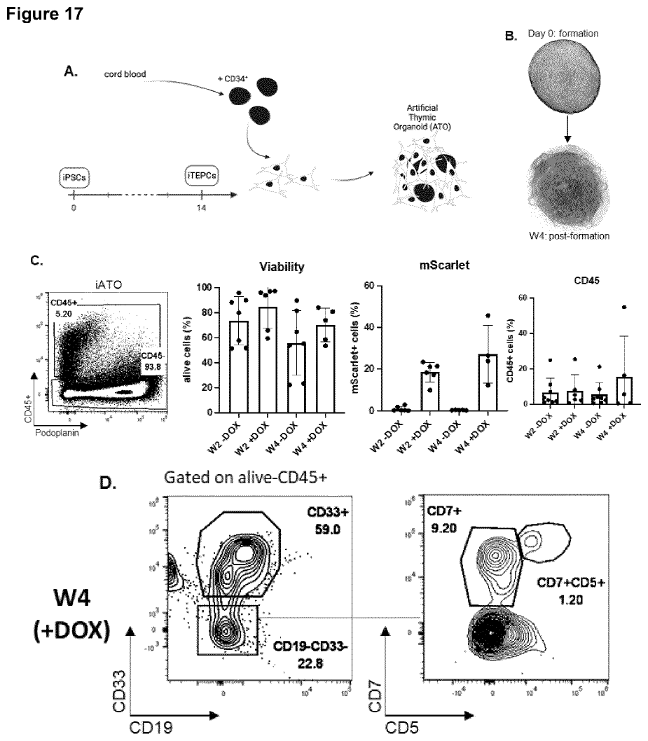

The pilot study provided important information to optimize a detailed FC panel to better characterize the immune cells generated in the ATO system (FIG. 17). We developed an iATO system where iTEPCs are combined with human CD34+ and cultured in an improved iATO medium containing SCF (see methods). Over the course of 4 weeks of culture (W4), defined morphological structures are observed (FIG. 17B). In addition, the iATO system exhibits robust cell survival throughout 4 weeks of culture and maintained the mScarlet expression (FIG. 17C). Although immune cells, identified by general CD45 expression, are present in low amounts, they remain stable over 4 weeks (FIG. 17C). Looking in more detail at the immune population that develops in the iATO system, we observed a large CD33+ population representing myeloid cells, as well as the emergence of CD5+CD7+ cells which define the early committed T-cell progenitors.

In Vivo Development of Mature CD4+ and CD8+ T Cells Following iTEPC Transplantation in Balb/C Nude Mice.

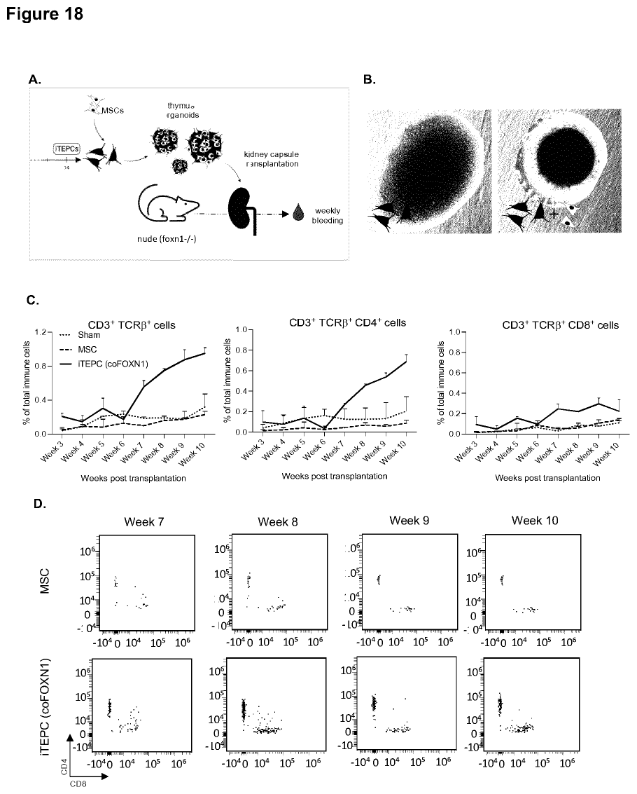

To assess the in vivo potential of iTEPCs to induce mature CD4+ and CD8+ T cells, we utilized Balb/C nude mice, which lack a thymus and consequently exhibit impaired T cell development but have otherwise normal hematopoietic cell function. We transplanted iTEPC organoids under the kidney capsule (FIG. 18A), employing a combined culture approach with mesenchymal stem cells (MSCs) to promote proper aggregation of iTEPCs and thymic spheroid formation (FIG. 18B). Mice were either subjected to sham treatment or transplanted with organoids containing MSCs only (n=6 per kidney) or with organoids containing iTEPC/MSCs (n=6 per kidney). Mature T cells, characterized by the presence of CD3+ TCRβ+CD4+ or CD3+TCRβ+ CD8+ markers, are expected to leave the thymus and circulate in the periphery. Blood monitoring over time revealed a significant increase in the frequency of total CD3+ TCRβ+ cells and mature CD4+ or CD8+ T cells in mice engrafted with iTEPCs (FIG. 18C, D). These results show that iTEPCs support the development of T cells into fully mature states.

In Vivo Detection of T Cell Progenitors in Humanized Mice Engrafted with iTEPCs

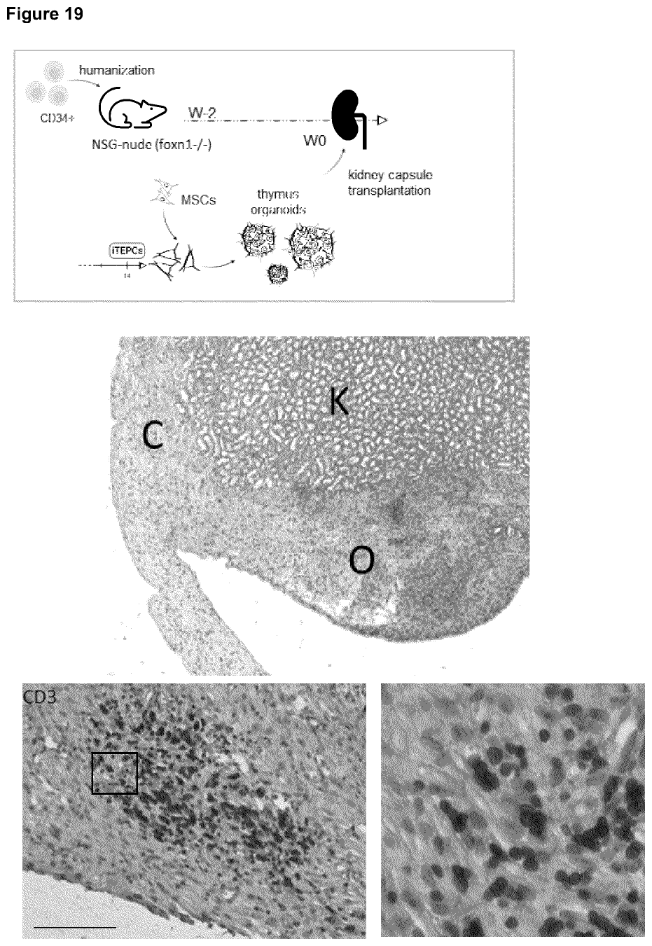

Next, we investigated whether iTEPCs could induce T cell development in an in vivo humanized setting. Briefly, mice were conditioned with busulfan two days before the adoptive transfer of human CD34+ hematopoietic progenitor cells (HPCs). After two weeks, organoids comprising iTEPCs, MSCs and/or primed CD34+ cells, were transplanted under the kidney capsule of humanized NSG-nude mice. The development of committed T cell progenitors was then assessed by performing immunohistochemistry (IHC) staining for CD3 on kidney samples at week 7 post-transplantation (FIG. 19). Human CD3+ cells were found within the organoid but not in the surrounding kidney capsule. These data provide proof of concept that our iTEPCs specifically support human T cell development within the organoids. Specifically, these findings demonstrate that our iTEPCs have the capacity to support thymocyte differentiation along the T cell development trajectory.

### REFERENCES

Abaci, Alperen, and Murat Guvendiren. “Designing decellularized extracellular matrix-based bioinks for 3D bioprinting.” Advanced healthcare materials 9.24 (2020): 2000734.
Brafman D A, Moya N, Allen-Soltero S, Fellner T, Robinson M, McMillen Z L, et al. Analysis of SOX2-expressing cell populations derived from human pluripotent stem cells. Stem cell reports. 2013 Nov. 19; 1 (5): 464-78.
Baik S, Jenkinson E J, Lane P J L, Anderson G, Jenkinson W E. Generation of both cortical and Aire (+) medullary thymic epithelial compartments from CD205 (+) progenitors. Eur J Immunol. 2013 March; 43 (3): 589-94.
Cereso N, Pequignot M O, Robert L, Becker F, De Luca V, Nabholz N, Rigau V, De Vos J, Hamel C P, Kalatzis V. Proof of concept for AAV2/5-mediated gene therapy in iPSC-derived retinal pigment epithelium of a choroideremia patient. Mol Ther Methods Clin Dev. 2014 Apr. 2; 1:14011. doi: 10.1038/mtm.2014.11
Chhatta, Amiet R., et al. “De novo generation of a functional human thymus from induced pluripotent stem cells.” Journal of Allergy and Clinical Immunology 144.5 (2019): 1416-1419.
Chen X, Janssen J M, Liu J, Maggio I, 't Jong A E J, Mikkers H M M, et al. In trans paired nicking triggers seamless genome editing without double-stranded DNA cutting. Nat Commun. 2017 September; 8 (1): 657.
D'Amour, Kevin A., et al. “Efficient differentiation of human embryonic stem cells to definitive endoderm.” Nature biotechnology 23.12 (2005): 1534-1541
Gras-Peña R, Danzl N M, Khosravi-Maharlooei M, Campbell S R, Ruiz A E, Parks C A, et al. Human stem cell-derived thymic epithelial cells enhance human T-cell development in a xenogeneic thymus. J Allergy Clin Immunol. 2022 May 1; 149 (5): 1755-71.
Green M D, Chen A, Nostro M-C, Schaniel C, Lemischka I R, Gouon-Evans V, et al. Generation of anterior foregut endoderm from human embryonic and induced pluripotent stem cells. Nat Biotechnol. 2011; 29 (3): 267-73.
Haunerdinger V, Moccia M D, Opitz L, Vavassori S, Dave H, Hauri-Hohl M M. Novel Combination of Surface Markers for the Reliable and Comprehensive Identification of Human Thymic Epithelial Cells by Flow Cytometry: Quantitation and Transcriptional Characterization of Thymic Stroma in a Pediatric Cohort. Front Immunol. 2021 Sep. 30; 12:4050.
Jiang W, Swiggard W J, Heufler C, Peng M, Mirza A, Steinman R M, et al. The receptor DEC-205 expressed by dendritic cells and thymic epithelial cells is involved in antigen processing. Nature. ms1995; 375 (6527): 151-5.
Kim, Jihoon, Bon-Kyoung Koo, and Juergen A. Knoblich. “Human organoids: model systems for human biology and medicine.” Nature Reviews Molecular Cell Biology 21.10 (2020): 571-584.
Leibel S L, Tseu I, Zhou A, Hodges A, Yin J, Bilodeau C, et al. Metabolomic profiling of human pluripotent stem cell differentiation into lung progenitors. iScience. 2022 Feb. 18; 25 (2).
Moroni, Lorenzo, et al. “Biofabrication strategies for 3D in vitro models and regenerative medicine.” Nature Reviews Materials 3.5 (2018): 21-37.
Otsuka, Ryo, et al. “Efficient generation of thymic epithelium from induced pluripotent stem cells that prolongs allograft survival.” Scientific reports 10.1 (2020): 224.
Parent A V., Russ H A, Khan I S, Laflam T N, Metzger T C, Anderson M S, et al. Generation of functional thymic epithelium from human embryonic stem cells that supports host T cell development. Cell Stem Cell. 2013 Aug. 1; 13 (2): 219-29.
Ramos, Stephan A., et al. “Generation of functional thymic organoids from human pluripotent stem cells.” Stem Cell Reports 18.4 (2023): 829-840.
Reeh K A G, Cardenas K T, Bain V E, Liu Z, Laurent M, Manley N R, et al. Ectopic TBX1 suppresses thymic epithelial cell differentiation and proliferation during thymus organogenesis. Development. 2014 Aug. 1; 141 (15): 2950-8.
Seet C S, He C, Bethune M T, Li S, Chick B, Gschweng E H, et al. Generation of mature T cells from human hematopoietic stem and progenitor cells in artificial thymic organoids. Nat Methods. 2017; 14 (5): 521-30.
Shakib S, Desanti G E, Jenkinson W E, Parnell S M, Jenkinson E J, Anderson G. Checkpoints in the Development of Thymic Cortical Epithelial Cells1. J Immunol. 2009 Jan. 1; 182 (1): 130-7.
Stoeckle C, Rota I A, Tolosa E, Haller C, Melms A, Adamopoulou E. Isolation of myeloid dendritic cells and epithelial cells from human thymus. J Vis Exp. 2013 September; (79): e50951.
Wang, Han, et al. “Improvement of cell survival during human pluripotent stem cell definitive endoderm differentiation.” Stem Cells and Development 24.21 (2015): 2536-2546
Zeleniak A, Wiegand C, Liu W, McCormick C, K. R, Alavi A, et al. De novo construction of T cell compartment in humanized mice engrafted with iPSC-derived thymus organoids. Nat Methods. 2022; 19 (10): 130
Žuklys S, Handel A, Zhanybekova S, Govani F, Keller M, Maio S, et al. Foxn1 regulates key target genes essential for T cell development in postnatal thymic epithelial cells. Nat Immunol. 2016 October; 17 (10): 1206-15.

1. A method for producing a three dimensional thymic like structure, the method comprising:
a) providing pluripotent cell(s) from a recipient with a nucleic acid molecule comprising a FOXN1 sequence, and b) inducing said pluripotent cell(s) to differentiate into thymic epithelium in a scaffold suitable for transplantation.

2. A method for producing a three dimensional thymic like structure, the method comprising:
a) providing pluripotent cell(s) from a recipient, and b) inducing said pluripotent cell(s) to differentiate into thymic epithelium in a scaffold suitable for transplantation, wherein a nucleic acid molecule comprising a FOXN1 sequence is provided to the pluripotent cell(s) from a recipient of a) or to cells of a differentiation stage in b) selected from definitive endoderm cells, anterior foregut endoderm cells, and pharyngeal pouch endoderm cells.

3. The method according to claim 1 or 2, wherein said scaffold comprises cells derived from the recipient and/or HLA-matched with such recipient.

4. A nucleic acid molecule comprising a FOXN1 sequence, wherein the nucleic acid further comprises nucleic acid sequences for introducing said FOXN1 sequence in a pluripotent cell.

5. The nucleic acid molecule of claim 4, wherein the nucleic acid further comprises nucleic acid sequences for introducing said FOXN1 sequence in a safe harbour locus of a pluripotent cell.

6. A vector comprising a nucleic acid molecule according to claim 4.

7. A vector comprising a nucleic acid molecule according to claim 5.

8. The vector of claim 7, wherein the nucleic acid sequences for introducing a FOXN1 sequence in a safe harbour locus of a pluripotent cell comprise homologous sequences to a safe harbour locus.

9. A vector according to any one of claims 7-8, wherein the safe harbour locus is AAVS1, CCR5, or humanRosa26.

10. A vector according to claim 6, wherein the vector remains episomal upon introduction to a pluripotent cell.

11. A viral vector comprising a vector according to any one of claims 6-9 and a viral capsid.

12. A viral vector according to claim 11 which is of lentiviral origin.

13. A method for producing a thymic epithelial progenitor cell (TEPC) from a pluripotent cell comprising the steps: providing said pluripotent cell with a nucleotide vector according to any one of claims 6-10, thereby obtaining a transduced cell, and subjecting said transduced cell to a differentiation protocol, optionally wherein the differentiation protocol comprises stepwise and timed modulation of key developmental pathways including the wnt, SHH, TGF, BMP and/or retinoic acid signaling pathways.

14. A method for producing a TEPC from a pluripotent cell comprising the steps: infecting a pluripotent cell with a viral vector according to any one of claims 11-12, thereby obtaining a transduced cell, and subjecting said transduced cell to a differentiation protocol, optionally wherein the differentiation protocol comprises stepwise and timed modulation of key developmental pathways including the wnt, SHH, TGF, BMP and/or retinoic acid signaling pathways.

15. The method for producing a TEPC from a pluripotent cell according to claim 13 or 14, wherein the pluripotent cell differentiates into TEPC via the intermediate stages of definitive endoderm cells, anterior foregut endoderm cells, and pharyngeal pouch endoderm cells and/or wherein the pluripotent cell differentiates into definitive endoderm cells and the differentiation of definitive endoderm to anterior foregut endoderm is induced when the percentage of SP CD117+ is at least 30% and the percentage of DP CD117+CD184+ cells is at most 70%.

16. The method for producing a TEPC from a pluripotent cell according to claim 15, wherein the differentiation of definitive endoderm to anterior foregut endoderm is induced by exposing the cells to dual TGFβ/BMP inhibition, in particular dual TGFβ/BMP4 inhibition.

17. The method for producing a TEPC from a pluripotent cell according to any one of claims 13-16, wherein FOXN1 expression is induced at the pharyngeal pouch endoderm stage.

18. A thymus epithelial progenitor cell (TEPC) comprising a FOXN1 encoding sequence integrated in its DNA in a safe harbour locus.

19. A TEPC according to claim 18, wherein the cells of the thymus organoid are of human origin.

20. A TEPC according to claim 18 or 19, wherein the safe harbour locus is AAVS1, CCR5, or humanRosa26.

21. A TEPC comprising a FOXN1 encoding sequence integrated in its DNA.

22. The TEPC according to claim 21, wherein the TEPC comprises said FOXN1 encoding sequence randomly integrated in its DNA.

23. A TEPC according to claim 18-22, wherein the FOXN1 encoding sequence is under the control of an inducible promoter.

24. A TEPC according to claim 23, wherein the inducible promoter is a tetracycline-inducible promoter (TET on or TET off promoter), an estrogen inducible promoter (e.g. a promoter inducible by estradiol or analogues thereof or by tamoxifen, a cumate-controlled promotor (e.g. VP16), a FKBP12 and/or cyclophilin inducible promoter, a FRAP (protein-protein interaction induced by small molecule) promoter, or a metallothionein promoter.

25. A method for inducing a thymic epithelial cell (TEC) from a TEPC as defined in claims 18-24, the method comprising contacting said TEPC with an autologous hematopoietic stem cell or autologous hematopoietic progenitor cell or an autologous common lymphoid progenitor or an autologous T-cell progenitor under conditions allowing for said induction, optionally wherein said contacting comprises co-culturing said TEPC with an autologous hematopoietic stem cell or an autologous common lymphoid progenitor or an autologous T-cell progenitor.

26. A TEC obtainable by the method of claim 25.

27. A thymus spheroid or organoid comprising at least one TEPC according to any one of claims 18-24 and/or at least one TEC according to claim 26.

28. A spheroid or organoid according to claim 27, further comprising supporting cells.

29. A spheroid or organoid according to claim 27, wherein the supporting cells comprise mesenchymal stromal cells (MSCs).

30. A spheroid or organoid according to claim 28 or 29, wherein the supporting cells comprise fibroblasts.

31. A spheroid or organoid according to any one of claims 27-30, wherein the supporting cells share their origin with the TEPCs and/or TECs.

32. A spheroid or organoid according to any one of claims 27-31, further comprising a supporting matrix that comprises Collagen type 1, hydrogels based on fibronectin, laminins, and other proteoglycans, alginate, hyaluronic acid-based gels, or Hystem, or a combination of at least any two thereof.

33. A spheroid or organoid according to claim 32, wherein the supporting matrix comprises 3D printed materials.

34. A thymus spheroid or organoid according to any one of claims 27-34, wherein the thymus spheroid or organoid has a medulla-like and/or cortex-like three-dimensional structure.

35. A spheroid or organoid according to any one of the claims 27-34 for use in cell therapy.

36. A spheroid or organoid according to any one of the claims 27-34, for use in the treatment of congenital and/or acquired diseases.

37. A spheroid or organoid according to any one of the claims 27-34, for use in the treatment of an immune deficiency.

38. A spheroid or organoid according to any one of the claims 27-34, for use in the treatment of a lack of a functional thymus.

39. A spheroid or organoid according to any one of the claims 27-34, for use in the treatment of DiGeorge syndrome, Otofaciocervical Syndrome, or CHARGE syndrome.

40. A spheroid or organoid according to any one of the claims 27-34, for use in the treatment of FOXN1 deficiency.

41. A spheroid or organoid according to any one of the claims 27-34, for use in the treatment of a lack of a fully functional immune repertoire as a result of chemotherapy.

42. A spheroid or organoid according to any one of the claims 27-34, for use in the treatment of a lack of a fully functional immune repertoire as a result of hematopoietic stem cell transplantation.

43. A spheroid or organoid according to any one of the claims 27-34, for use in the treatment of a lack of a functional thymus as a result of thymectomy.

44. A spheroid or organoid according to any one of the claims 27-34, for use in the treatment of a lack of a functional thymus as a result of thymus involution.

45. A spheroid or organoid for use according to any one of claims 35 to 44, wherein the spheroid or organoid is implanted in skeletal muscle.

46. A spheroid or organoid for use according to claim 45, wherein the skeletal muscle is a quadriceps muscle.

47. A spheroid or organoid for use according to any one of the claims 45-46, wherein said skeletal muscle shares its origin with said spheroid or organoid.

48. The nucleic acid molecule comprising a FOXN1 sequence according to claim 4 or 5, or the vector or viral vector according to any one of claims 6 to 12, wherein said FOXN1 sequence is codon optimized for human codon usage, and wherein said codon optimization comprises removal of at least one CpG island and/or at least one cryptic splice site.

49. A method of producing at least one T cell or T lineage cell, wherein the method comprises the steps of:
a) providing a spheroid or organoid as specified in any one of claims 27 to 34 comprising at least one thymus epithelial cell (TEC) and/or thymus epithelial progenitor cell (TEPC),
b) contacting the spheroid or organoid of a) with at least one hematopoietic progenitor cell or T lineage cell, thereby producing at least one T cell or T lineage cell.
